(19) United States
(12) Patent Application Publication
(10) Pub. No.: US 2007/0059722 A1
(43) Pub. Date: Mar. 15, 2007

(54) Novel genes and markers associated to type 2 diabetes mellitus

(75) Inventors: Jukka T. Salonen, Kuopio (FI); Juha-Matti Aalto, Siilinjarvi (FI); Ricardo Fuentes, Kuopio (FI); Outi Kontkanen, Kuopio (FI); Mia Pirskanen, Kuopio (FI); Pekka Uimari, Kuopio (FI)

Correspondence Address: BIRCH STEWART KOLASCH & BIRCH, PO BOX 747, FALLS CHURCH, VA, 22040-0747, US

(73) Assignee: Oy Jurilab Ltd, Kuopio (FI)

(21) Appl. No.: 11/325,330

(22) Filed: Jan. 5, 2006

(30) Foreign Application Priority Data
Jan. 5, 2005 (FI) 20050011
Dec. 23, 2005 (WO) PCT/FI05/50486

(51) Int. Cl. C12Q 1/68; G06F 19/00

(52) U.S. Cl. 435/6; 702/20

(57) ABSTRACT

Genes, SNP markers and haplotypes of susceptibility or predisposition to T2D and subdiagnosis of T2D are disclosed. Methods for diagnosis, prediction of clinical course and efficacy of treatments for T2D using polymorphisms in the T2D risk genes are also disclosed. The genes, gene products and agents of the invention are also useful for monitoring the effectiveness of prevention and treatment of T2D. Kits are also provided for the diagnosis, selecting treatment and assessing prognosis of T2D.

### BACKGROUND OF THE INVENTION

1. Field of the Invention

The present invention relates generally to the field of diagnosis of diabetes mellitus (DM). More particularly, it provides a method of diagnosing or detecting a predisposition or propensity or susceptibility for type 2 diabetes mellitus (T2D). Specifically, the invention is directed to a method that comprises the steps of providing a biological sample of the subject to be tested and detecting the presence or absence of one or several genomic single nucleotide polymorphism (SNP) markers in the biological sample. Furthermore, the invention utilises both genetic and phenotypic information as well as information obtained by questionnaires to construct a score that provides the probability of developing T2D. In addition, the invention provides a kit to perform the method. The kit can be used to set an etiology-based diagnosis of T2D for targeting of treatment and preventive interventions, such as dietary advice as well as stratification of the subject in clinical trials testing drugs and other interventions. The kit can also be used for the prediction of clinical course and efficacy of treatments for T2D.

2. Description of Related Art

Public Health Significance of T2D

The term diabetes mellitus (DM) (ICD/10 codes E10-E14) describes several syndromes of abnormal carbohydrate metabolism that are characterized by hyperglycemia. It is associated with a relative or absolute impairment in insulin secretion, along with varying degrees of peripheral resistance to the action of insulin. The chronic hyperglycemia of diabetes is associated with long-term damage, dysfunction, and failure of various organs, especially the eyes, kidneys, nerves, heart, and blood vessels (ADA, 2003). T2D is characterized by adult onset insulin resistance and a rise in blood sugar concentration.

In 2000, there were approximately 171 million people, worldwide, with diabetes. The number of people with diabetes will expectedly more than double over the next 25 years, to reach a total of 366 million by 2030 (WHO/IDF, 2004). Most of this increase will occur as a result of a 150% rise in developing countries. This suggests the role of relatively modern environmental or behavioral risk factors such as high caloric intake or sedentary lifestyle. However, ethnic differences in the incidence and prevalence of T2D and the enrichment of T2D in families suggest heritable risk factors to play a major role. In the USA, there are over 15 million diabetics and 15 million people with impaired glucose tolerance. Almost one million Americans become diabetic annually.

The two main contributors to the worldwide increase in prevalence of diabetes are population ageing and urbanization, especially in developing countries, with the consequent increase in the prevalence of obesity (WHO/IDF, 2004). Currently more than 1 billion adults are overweight - and at least 300 million of them are clinically obese. Current obesity levels range from below 5% in China, Japan and certain African nations, to over 75% in urban Samoa. The prevalence of obesity is 10-25% in Western Europe and 20-27% in the Americas (WHO, 2004).

In 2000, 3.2 million people died from complications associated with diabetes. Diabetes has become one of the major causes of premature illness and death in most countries, mainly through the increased risk of cardiovascular disease (CVD). Diabetes is a leading cause of blindness, amputation and kidney failure. These complications account for much of the social and financial burden of diabetes (WHO/IDF, 2004).

Because of the chronic nature of T2D, the severity of its complications and the means required to control them, diabetes is a costly disease, not only for the affected individual and his/her family, but also for the health authorities. In the US direct medical and indirect expenditures attributable to diabetes in 2002 were estimated at $132 billion. Direct medical expenditures alone totalled $91.8 billion and comprised $23.2 billion for diabetes care, $24.6 billion for chronic complications attributable to diabetes, and $44.1 billion for excess prevalence of general medical conditions. Attributable indirect expenditures resulting from lost workdays, restricted activity days, mortality, and permanent disability due to diabetes totalled $39.8 billion (ADA, 2003).

Classification of DM

According to the new etiologic classification of DM, four categories are differentiated: type 1 diabetes (T1D), type 2 diabetes (T2D), other specific types, and gestational diabetes mellitus (ADA, 2003). In the United States, Canada, and Europe, over 80% of cases of diabetes are due to T2D, 5 to 10% to TID, and the remainder to other specific causes.

In T1D, formerly known as insulin-dependent (IDDM), the pancreas fails to produce the insulin which is essential for survival. This form develops most frequently in children and adolescents, but is being increasingly diagnosed later in life. T2D, formerly named non-insulin-dependent (NIDDM), results from the body's inability to respond properly to the action of insulin produced by the pancreas. T2D occurs most frequently in adults, but is being noted increasingly in adolescents as well (WHO, 2004).

Pathophysiology of T2D

The causes of T2D are multi-factorial and include both genetic and environmental elements that affect beta cell function and tissue insulin sensitivity (muscle, liver, adipose tissue, pancreas). Although there is considerable debate as to the relative contributions of beta-cell dysfunction and reduced insulin sensitivity to the pathogenesis of diabetes, it is generally agreed that both of these factors play important roles (Scheen A J, 2003).

Insulin Resistance

Insulin resistance is probably the first defect in T2D and begins many years before the onset of symptoms or developing a blood glucose level high enough to make the diagnosis (Martin B C et al, 1992). Insulin resistance occurs in the peripheral cells of the body (primarily muscle and fat cells) and the liver. It is caused by genetic factors (see below) as well as environmental factors. The environmental factors include aging, sedentary lifestyle, and central obesity.

Normally, insulin-signalling proceeds by insulin binding to its cell surface receptor protein, which activates the intrinsic tyrosine kinase activity of the receptor (Saltiel A R and Kahn C R, 2001). The activated receptor kinase then phosphorylates protein substrates within the cell on tyrosine residues. These tyrosine phosphorylated insulin receptor substrate proteins act as docking sites, binding other cellular signalling molecules that serve as adapters in the formation of complexes of intracellular signaling proteins. Downstream from these signalling complexes, the various cellular actions of insulin on glucose and lipid metabolism as well as cell growth are stimulated.

Recent research has delineated specific mechanisms whereby excess adiposity can influence the normal cellular actions of insulin and contribute to insulin resistance. Among the factors released by adipose tissue free fatty acids (FFA) are elevated in persons with increased visceral adipose tissue (Boden G, 2001; Shulman G I, 2000). When FFAs are elevated for a prolonged amount of time, they have a direct effect on insulin action in skeletal muscle tissue and liver, reducing the normal responses to insulin to promote glucose uptake and to suppress hepatic glucose output, respectively (Boden G, 2001; Shulman G I, 2000). In both of these tissues, FFA increase cellular levels of acyl-CoA derivatives, which leads to an increase in the activity of cellular signalling molecules termed serine kinases that oppose the normal tyrosine phosphorylation cascade of the insulin receptor (Zick Y, 2001). The increased intracellular lipid accumulation that occurs in obese subjects as ectopic fat—that is, triglyceride stored in the target organs themselves rather than in a benign adipose depot—is another important source of intracellular acyl-CoA molecules that can affect normal insulin signal transduction (Ravussin E and Smith S R, 2002). Other proteins secreted by adipose tissue, including the important inflammatory mediators interleukin-6 (IL-6) and tumor necrosis factor alpha (TNFα), have adverse effects on energy metabolism and insulin sensitivity in liver and muscle and play key roles in the development of insulin resistance in obesity (Dandona P et al, 2004). Adiponectin is a recently described plasma protein secreted uniquely from adipose tissue. Unlike TNFα and FFA, whose plasma levels are increased in visceral obesity, the levels of adiponectin are reduced in obese subjects (Berg A H et al, 2002). Studies of the physiologic role of adiponectin have provided evidence that it enhances insulin action and improves sensitivity as well as having anti-inflammatory protective effects on the vascular endothelium. Adiponectin improves the plasma clearance of FFA, glucose, and triglycerides and suppresses hepatic glucose production (Berg A H et al, 2002). In liver, skeletal muscle, and adipose tissue, the mechanism of action of adiponectin has been shown to involve adenosine monophosphate activated protein kinase (AMP kinase) a signalling kinase implicated in the insulin dependent uptake of glucose into skeletal muscle and also in the cellular mechanism of action of metformin (Wu X et al, 2001;Yamauchi T et al, 2002; Tomas E et al, 2002). Also, consistent with the influence of visceral adiposity on insulin resistance and the metabolic syndrome, the regulation of circulating levels of adiponectin appears to be at the level of omental adipose tissue as opposed to the subcutaneous depot (Motoshima H et al, 2002).

Insulin Deficiency

As insulin resistance develops, the beta cells increase insulin production to compensate and maintain the blood glucose level in the narrow range needed for normal body function. If insulin resistance persists or increases over time (usually 3-5 years) the beta cells will begin to fail. When insulin resistance persists but insulin secretion decreases and blood glucose levels begin to rise, true diabetes has developed (Guthrie R A and Guthrie D W, 2004). The pattern of beta cell function loss is an initial defect in acute or first-phase insulin secretion, followed by a decreasing maximal capacity of insulin secretion. Last, a defective steady-state and basal insulin secretion develops, leading to almost complete beta-cell failure requiring insulin treatment. Because of the reciprocal relation between insulin secretion and insulin sensitivity, valid representation of beta cell function requires interpretation of insulin responses in the context of the prevailing degree of insulin sensitivity (Scheen A J, 2004). Evidence of the progressive loss of beta cell function may include altered conversion of proinsulin to insulin, changes in pulsed and oscillatory insulin secretion, and quantitative reductions in insulin release. Potential underlying mechanisms are glucose toxicity, lipotoxicity, poor tolerance of increased secretory demand, and a reduction in beta-cell mass (Buchanan T A, 2003). The roles of glucose and fatty acids in altering the function of various cell types in diabetes, in particular that of the beta cells is summarized in the “glucolipotoxicity” hypothesis (Prentki M et al, 2002). It is suggested that either hyperglycemia alone or elevated circulating FFAs alone should not be so detrimental to a cell for the simple reason that when glucose levels alone are high, glucose is oxidized, and when FFAs alone are high, then they are oxidized instead of glucose. However, when both glucose and FFA levels are high they may progressively alter the function of various cell types. Under this condition, FFA-derived long-chain acyl-CoA esters (FACoAs) are high, and furthermore they cannot be oxidized because glucose-derived malonyl-CoA is also elevated. Malonyl-CoA is a metabolic signalling molecule that regulates lipid partitioning (the relative fluxes of FFA oxidation and esterification) through its inhibitory action on carnitine palmitoyltransferase-1 (CPT1), which catalyzes the rate-limiting step of the mitochondrial β-oxidation of fatty acids. As a result, FACoAs accumulate in the cytoplasm and could, for example, promote beta-cell apoptosis. Indeed, it is now realized that, in the vast majority of T2D cases, there is a decreased beta-cell mass caused by a marked increase in beta-cell apoptosis that outweighs rates of beta-cell mitogenesis and neogenesis. Recent advances have implicated signal transduction via insulin receptor substrate-2 (IRS-2) and downstream via protein tyrosine kinase 2 beta (PTK2B) as critical to the control of beta cell survival (Dickson L M and Rhodes C J, 2004).

T2D: a Polygenic Disease

T2D has a complex mode of inheritance, as corroborated by family studies indicating major roles of both genetic susceptibility and the environment. There was significant familial aggregation of T2DM and related phenotypes. Generally, the sibling of a patient with T2D has a four to six-fold higher risk of developing the disease (30%-40%) than does an unrelated individual (7%) (Florez J C et al, 2003). A strong genetic influence has also been suggested by a greater concordance rate for type 2 diabetes in monozygotic (MZ) compared with dizygotic (DZ) twin pairs. Rates of concordance of T2D are much higher for monozygotic twins as compared to dizygotic twins. It has been reported that 90% of identical twin pairs were concordant for T2D if followed for long enough (Barnett A H et al, 1981). A concordance rate for T2D of 43% in Danish dizygotic twins as compared to 63% in monozygotic twins has also been reported (Poulsen et al, 1999). The heritability of T2D, glucose intolerance and insulin secretion has been estimated to be 60-80% (Lowe 2001, Lehtovirta et al 2005).

Known monogenic forms of diabetes are classified in two categories: genetic defects of the beta cell and genetic defects in insulin action (ADA, 2003). The diabetes forms associated with monogenetic defects in beta cell function are frequently characterized by onset of hyperglycemia at an early age (generally before age 25 years). They are referred to as maturity-onset diabetes of the young (MODY) and are characterized by impaired insulin secretion with minimal or no defects in insulin action (Herman W H et al, 1994; Clement K et all, 1996; Byrne M M et al, 1996). They are inherited in an autosomal dominant pattern. Abnormalities at three genetic loci on different chromosomes have been identified to date. The most common form is associated with mutations on chromosome 12q in the locus of a hepatic transcription factor referred to as hepatocyte nuclear factor (HNF)-1α (Vaxillaire M et all, 1995; Yamagata K et al, 1996). A second form is associated with mutations in the locus of the glucokinase gene on chromosome 7p and results in a defective glucokinase molecule (Froguel P et al, 1992; Vionnet N et al, 1992). Glucokinase converts glucose to glucose-6-phosphate, the metabolism of which, in turn, stimulates insulin secretion by the beta cell. Because of defects in the glucokinase gene, increased plasma levels of glucose are necessary to elicit normal levels of insulin secretion. A third form is associated with a mutation in the HNF-4α gene on chromosome 20q (Bell GI et al, 1991; Yamagata K et al, 1996). HNF-4α is a transcription factor involved in the regulation of the expression of HNF-1α. Point mutations in mitochondrial DNA can cause DM primarily by impairing pancreatic beta cell function (Reardon W et al, 1992; van den Ouwenland J M W et al, 1992; Kadowaki T et al, 1994). There are unusual causes of diabetes that result from genetically determined abnormalities of insulin action. The metabolic abnormalities associated with mutations of the insulin receptor may range from hyperinsulinemia and modest hyperglycemia to severe diabetes (Kahn C R et al, 1976; Taylor S I, 1992).

In most cases, T2D results from a complex interaction of genetic, environmental, and demographic factors. Improved techniques of genetic analysis, especially candidate gene association studies and genome wide linkage analysis (genome wide scan, GWS), have enabled a search for genes that contribute to the development of T2D in the population.

No major single genes explaining the development of T2D have been identified. However, studies have demonstrated associations between various metabolic defects underlying the development of type 2 diabetes and polymorphisms in several susceptibility genes (e.g., PPARγ and PGC-1). Although more than a hundred candidate genes have been evaluated for T2D, only a handful have been widely replicated. The association of PPARγ with T2D is widely reproduced (Deeb S S et al, 1998; Hara K et al, 2000; Altshulert D et al, 2000; Mori H et al, 2001), and that of KCNJ1 (Hani E H et al, 1998; Gloyn A L et al, 2001; Gloyn A L et al, 2003), ABCC8 (Inoue H, 1996; Hani E H et al, 1997; Hansen T et al, 1998), GCGR (Hager J et al, 1995; Gough S C et al, 1995), GCK (Chiu K C et al, 1992; McCarthy M I et al, 1994; Takekawa K et al, 1994) and SLC2A1 (Li S R et al, 1988; Tao T et al, 1995; Pontiroli A E et al, 1996) have now been seen by multiple groups. An example of recent, still uncorroborated, findings is ARNT gene, a transcription factor essential for embryonic development (Gunton et al 2005).

Gene-environment interactions have been found between PPARG and birth weight affecting adult insulin sensitivity (Eriksson J G et al, 2002) and between PPARG and dietary fat intake influencing adult BMI (Luan J et al, 2001; Memisoglu A et al, 2003). A gene-gene interaction has also been found between PPARG and FABP4 affecting adult insulin sensitivity and body composition (Damcott C M et al, 2004).

To date more than 30 GWSs have been reported to identify loci for T2D. Linked loci with at least suggestive LOD scores have been observed on every chromosome. Perhaps most striking is the lack of consistently linked loci. Demenais F et al, 2003 applying the genome-search meta-analysis method (GSMA) to 4 published genome-wide scans of T2D from Caucasian populations (GIFT consortium, Finland, Sweden, UK and France) found evidence of susceptibility regions for T2D on chromosomes 1p13.1-q22, 2p22.1-p13.2, 6q21-q24.1, 12q21.1-q24.12, 16p12.3-q11.2 and 17p11.2-q22, which had modest or non-significant linkage in each individual study. This may serve to illustrate the heterogeneity of human T2D as well as the potential shortcomings of attempting to compare studies using different methodologies.

Opportunity for Population Genetics

Developments in GWS and sequencing technology and methods of data analysis render now possible the attempt to identify liability genes in complex, multifactorial traits, and to dissect out with new precision the role of genetic predisposition and environment/life style factors in these disorders. Genetic and environmental effects vary over the life span, and only longitudinal studies in genetically informative data sets permit the study of such effects. A major advantage of population genetics approaches in disease gene discovery over other methodologies is that it will yield diagnostic markers which are valid in humans.

Identification of genes causing the major public health problems such as T2D is now enabled by the following recent advances in molecular biology, population genetics and bioinformatics: 1. the availability of new genotyping platforms that will dramatically lower operating cost and increase throughputs; 2. the application of genome scans using dense marker maps (>100.000 markers); 3. data analysis using new powerful statistical methods testing for linkage disequilibrium using haplotype sharing analysis, and 4. the recognition that a smaller number of genetic markers than previously thought is sufficient for genome scans in genetically homogeneous populations.

Traditional GWS using microsatellite markers with linkage analyses have not been successful in finding genes causing common diseases. The failure has in part been due to too small a number of genetic markers used in GWS, and in part due to too heterogeneous study populations. With the advancements of the human genome project and genotyping technology, the first dense marker maps have recently become available for mapping the entire human genome. The microarrays used by Jurilab include probes for over 100 000 SNP markers. These SNPs form a marker map covering, for the first time, the entire genome tightly enough for the discovery of the majority of disease genes causing T2D.

Genetic Homogeneity of the East Finland Founder Population

Finns descend from two human immigration waves occurring about 4,000 and 2,000 years ago, respectively. Both Y-chromosomal haplotypes and mitochondrial sequences show low genetic diversity among Finns compared with other European populations and confirm the long-standing isolation of Finland (Sajantila A et al, 1996). During King Gustavus of Vasa (1523-1560) over 400 years ago, internal migrations created regional subisolates, the late settlements (Peltonen L et al, 1999). The most isolated of these are the East Finns.

The East Finnish population is the most genetically-homogenous population isolate known that is large enough for effective gene discovery program. The reasons for homogeneity are: the young age of the population (fewer generations); the small number of founders; long-term geographical isolation; and population bottlenecks because of wars, famine and fatal disease epidemics.

Owing to the genetic homogeneity of the East Finland population there are fewer mutations in important disease predisposing genes and the affected individuals share similar genetic background. Because of the stronger linkage disequilibrium (LD), fewer SNPs and fewer subjects are needed for GWS than in other populations.

### SUMMARY OF THE INVENTION

The present invention relates to single nucleotide polymorphism (SNP) markers, combinations of such markers and haplotypes associated with altered risk of T2D and genes associated with T2D within or close to which said markers or haplotypes are located. Said SNP markers may be associated either with increased T2D risk or reduced T2D risk i.e. protective of T2D. The “prediction” or risk implies here that the risk is either increased or reduced.

Thus the present invention provides individual SNP markers associated with T2D and combinations of SNP markers and haplotypes in genetic regions associated with T2D, genes previously known in the art, but not known to be associated with T2D, methods of estimating susceptibility or predisposition of an individual to T2D, methods of determining the molecular subtype of T2D as well as methods for prediction of clinical course and efficacy of treatments for T2D using polymorphisms in the T2D risk genes. Accordingly the present invention provides novel methods and compositions based on the disclosed T2D associated SNP markers, combinations of SNP markers, haplotypes and genes.

The invention further relates to a method for estimating susceptibility or predisposition of an individual to T2D comprising the detection of the presence of SNP markers and haplotypes or an alteration in expression of a T2D risk gene set forth in tables I through 5, as well as alterations in the polypeptides encoded by the said T2D risk genes. The alterations may be quantitative, qualitative, or both.

The invention yet further relates to a method for estimating susceptibility or predisposition of an individual to T2D. The method for estimating susceptibility or predisposition of an individual to T2D is comprised of detecting the presence of at-risk haplotypes in an individual's nucleic acid.

The invention further relates to a kit for estimating susceptibility to T2D in an individual comprising wholly or in part: amplification reagents for amplifying nucleic acid fragments containing SNP markers, detection reagents for genotyping SNP markers and interpretation software for data analysis and risk assessment.

In one aspect, the invention relates to methods of diagnosing a predisposition to T2D. The methods of diagnosing a predisposition to T2D in an individual include detecting the presence of SNP markers predicting T2D, as well as detecting alterations in expression of genes which are associated with said markers. The alterations in expression can be quantitative, qualitative, or both.

A further object of the present invention is a method of identifying the risk of T2D by detecting SNP markers in a biological sample of the subject. The information obtained from this method can be combined with other information concerning an individual, e.g. results from blood measurements, clinical examination and questionnaires. The blood measurements include but are not restricted to the determination of plasma or serum cholesterol and high-density lipoprotein cholesterol. The information to be collected by questionnaire includes information concerning gender, age, family and medical history such as the family history of obesity and diabetes. Clinical information collected by examination includes e.g. information concerning height, weight, hip and waist circumference and other measures of adiposity and obesity.

The methods of the invention allow the accurate diagnosis of T2D at or before disease onset, thus reducing or minimizing the debilitating effects of T2D. The method can be applied in persons who are free of clinical symptoms and signs of T2D, in those who have family history of T2D or obesity or in those who have elevated level or levels of risk factors of T2D or obesity.

The invention further provides a method of diagnosing susceptibility to T2D in an individual. This method comprises screening for at-risk haplotypes that predict T2D that are more frequently present in an individual susceptible to T2D, compared to the frequency of its presence in the general population, wherein the presence of an at-risk haplotype is indicative of a susceptibility to T2D. The “at-risk haplotype” may also be associated with a reduced rather than increased risk of T2D. An “at-risk haplotype” is intended to embrace one or a combination of haplotypes described herein over the markers that show high correlation to T2D. Kits for diagnosing susceptibility to T2D in an individual are also disclosed.

Those skilled in the art will readily recognize that the analysis of the nucleotides present in one or several of the SNP markers of this invention in an individual's nucleic acid can be done by any method or technique capable of determining nucleotides present in a polymorphic site. As it is obvious in the art the nucleotides present in SNP markers can be determined from either nucleic acid strand or from both strands.

The major application of the current invention involves prediction of those at higher risk of developing T2D. Diagnostic tests that define genetic factors contributing to T2D might be used together with or independent of the known clinical risk factors to define an individual's risk relative to the general population. Better means for identifying those individuals at risk for T2D should lead to better preventive and treatment regimens, including more aggressive management of the risk factors for T2D such as obesity and of the risk factors for sequelae of T2D such as cigarette smoking, hypercholesterolemia, elevated LDL cholesterol, low HDL cholesterol, elevated BP, obesity, lack of physical activity, and inflammatory components as reflected by increased C-reactive protein levels or other inflammatory markers. Information on genetic risk may be used by physicians to help convince particular patients to adjust life style (e.g. to stop smoking, to reduce caloric intake, to increase exercise). Finally, preventive measures aimed at lowering blood pressure such as reduction of weight, intake of salt and alcohol can be both better motivated to the patients who are at an elevated risk of T2D and selected on the basis of the molecular subdiagnosis of T2D.

A further object of the invention is a method for molecular diagnosis of T2D. The genetic etiology of T2D in an individual will provide information of the molecular etiology of T2D. When the molecular etiology is known, the therapy can be selected on the basis of this etiology. For example, the drug that is likely to be effective, i.e. blood glucose lowering, can be selected without trial and error.

A further object of the invention is to provide a method for the selection of human subjects for studies testing antidiabetic effects of drugs. Another object of the invention is a method for the selection of subjects for clinical trials testing antidiabetic drugs.

Still another object of the invention is to provide a method for prediction of clinical course and efficacy of treatments for T2D using polymorphisms in the T2D risk genes. The genes, gene products and agents of the invention are also useful for treating T2D, for monitoring the effectiveness of their treatment, and for drug development. Kits are also provided for the diagnosis, treatment and prognosis of T2D.

### DETAILED DESCRIPTION OF THE INVENTION

Methods of Therapy

The present invention discloses novel methods for the prevention and treatment of T2D.

The term, “treatment” as used herein, refers not only to ameliorating symptoms associated with the disease, but also preventing or delaying the onset of the disease, and also lessening the severity or frequency of symptoms of the disease, preventing or delaying the occurrence of a second episode of the disease or condition; and/or also lessening the severity or frequency of symptoms of the disease or condition.

In particular, the invention relates to methods of treatment for T2D or susceptibility to T2D (for example, for individuals in an at-risk population such as those described herein); as well as to methods of treatment for manifestations and subtypes of T2D.

The present invention encompasses methods of treatment (prophylactic and/or therapeutic) for T2D, such as individuals in the target populations described herein, using a T2D therapeutic agent. A “T2D therapeutic agent” is an agent that alters (e.g., enhances or inhibits) enzymatic activity or function of a T2D risk affecting polypeptide, and/or expression of a T2D risk gene as described herein. Useful therapeutic agents can alter a T2D susceptibility polypeptide activity or function and/or expression of a disease susceptibility gene by a variety of means, such as, for example, by altering translation rate of a T2D susceptibility polypeptide encoding mRNA; by altering the transcription rate of the T2D risk gene; by altering posttranslational processing of a T2D susceptibility polypeptide; by interfering with a T2D susceptibility polypeptide activity and/or function (e.g., by binding to a T2D susceptibility polypeptide); by altering stability of a T2D susceptibility polypeptide; by altering the transcription rate of splice variants of a T2D risk gene or by inhibiting or enhancing the elimination of a T2D susceptibility polypeptide from target cells, organs and/or tissues.

Representative T2D therapeutic agents comprise the following: (a) nucleic acids, fragments, variants or derivatives of T2D associated genes described in this invention, nucleic acids encoding a T2D susceptibility polypeptide or an active fragment or a derivative thereof and nucleic acids modifying the expression of said T2D genes (e.g. antisense polynucleotides, catalytically active polynucleotides (e.g. ribozymes and DNAzymes), molecules inducing RNA interference (RNAi) and micro RNA), and vectors comprising said nucleic acids; (b) T2D susceptibility polypeptides, active fragments, variants or derivatives thereof, binding agents of T2D susceptibility polypeptides; peptidomimetics; fusion proteins or prodrugs thereof, antibodies (e.g., an antibody to a mutant T2D susceptibility polypeptide, or an antibody to a non-mutant T2D susceptibility polypeptide, or an antibody to a particular variant encoded by a T2D risk gene, as described above) and other polypeptides (e.g., T2D susceptibility receptors, active fragments, variants or derivatives thereof); (c) metabolites of T2D susceptibility polypeptides or derivatives thereof; (d) small molecules and compounds that alter (e.g., inhibit or antagonize) a T2D risk gene expression, activity and/or function of a T2D risk gene encoded polypeptide, or activity and/or function of a T2D gene related metabolic pathway and; (e) small molecules and compounds that alter (e.g. induce or agonize) a T2D risk gene expression, activity and/or function of a T2D risk gene encoded polypeptide, or activity and/or function of a T2D gene related metabolic pathway.

More than one T2D therapeutic agent can be used concurrently, if desired. The therapy is designed to alter (e.g., inhibit or enhance), replace or supplement activity and/or function of one or several T2D polypeptides or related metabolic pathways in an individual. For example, a T2D therapeutic agent can be administered in order to upregulate or increase the expression or availability of a T2D risk gene or a specific variant of a T2D susceptibility gene or, conversely, to downregulate or decrease the expression or availability of a T2D risk gene or a specific variant of a T2D risk gene. Upregulation or increasing expression or availability of a native T2D risk gene or a particular variant of a T2D susceptibility gene could interfere with or compensate for the expression or activity of a defective gene or variant; downregulation or decreasing expression or availability of a native T2D risk gene or a particular splicing variant of a T2D susceptibility gene could minimize the expression or activity of a defective gene or the particular variant and thereby minimize the impact of the defective gene or the particular variant.

The T2D therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a T2D susceptibility polypeptide, fragment, variant or derivative thereof), either by itself or included within a vector, can be introduced into cells of an individual affected by T2D using variety of experimental methods described in the art, so that the treated cells start to produce native T2D susceptibility polypeptide. Thus, cells which, in nature, lack of a native T2D risk gene expression and activity, or have abnormal T2D risk gene expression and activity, can be engineered to express a T2D susceptibility polypeptide or an active fragment or a different variant of said T2D susceptibility polypeptide. Genetic engineering of cells may be done either “ex vivo ” (i.e. suitable cells are isolated and purified from a patient and re-infused back to the patient after genetic engineering) or “in vivo” (i.e. genetic engineering is done directly to a tissue of a patient using a vehicle).

Alternatively, in another embodiment of the invention, a nucleic acid of the invention; a nucleic acid complementary to a nucleic acid of the invention; or a portion of such a nucleic acid (e.g., a polynucleotide), can be used in “antisense” therapy, in which a nucleic acid (e.g., a polynucleotide) which specifically hybridizes to the mRNA and/or genomic DNA of a T2D risk gene is administered in a pharmaceutical composition to the target cells or said nucleic acid is generated “in vivo”. The antisense nucleic acid that specifically hybridizes to the mRNA and/or DNA inhibits expression of the T2D susceptibility polypeptide, e.g., by inhibiting translation and/or transcription. Binding of the antisense nucleic acid can be due to conventional base pairing, or, for example, in the case of binding to DNA duplexes, through specific interaction in the major groove of the double helix.

In a preferred embodiment nucleic acid therapeutic agents of the invention are delivered into cells that express one or several T2D risk genes. A number of methods including, but not limited to, the methods known in the art can be used for delivering a nucleic acid to said cells. For example, a vector can be introduced in vivo such that it is taken up by a cell and directs the transcription of a RNA molecule, which induces RNA interference in the cell. Such a vector can remain episomal or become chromosomally integrated, and as long as it can be transcribed to produce the desired RNA molecules it will modify the expression of a T2D risk gene. Such vectors can be constructed by various recombinant DNA technology methods standard in the art.

The expression of an endogenous T2D risk gene can be also reduced by inactivating or “knocking out” a T2D risk gene or its promoter using targeted homologous recombination methods described in the art. Alternatively, expression of a functional, non-mutant T2D risk gene can be increased using a similar method: targeted homologous recombination can be used to replace a non-functional T2D risk gene with a functional form of the said gene in a cell.

In yet another embodiment of the invention, other T2D therapeutic agents as described herein can also be used in the treatment or prevention of T2D. The therapeutic agents can be delivered in a pharmaceutical composition, they can be administered systemically, or can be targeted to a particular tissue. The therapeutic agents can be produced by a variety of means, including chemical synthesis, cell culture and recombinant techniques (e.g. with transgenic cells and animals). Therapeutic agents can be isolated and purified to fulfill pharmaceutical requirements using standard methods described in the art.

A combination of any of the above methods of treatment (e.g., administration of non-mutant T2D susceptibility polypeptide in conjunction with RNA molecules inducing RNA interference targeted to the mutant T2D susceptibility mRNA) can also be used.

In the case of pharmaceutical therapy, the invention comprises compounds, which enhance or reduce the activity and/or function of one or several polypeptides encoded by T2D susceptibility genes set forth in table 12. The treatment may also enhance or reduce the expression of one or several genes selected from T2D susceptibility genes set forth in table 12.

In another embodiment of the invention, pharmaceutical therapy of the invention comprises compounds, which enhance or reduce the activity and/or function of one or several biological networks and/or metabolic pathways related to T2D susceptibility genes, proteins or polypeptides. The treatment may also enhance or reduce the expression of one or several genes in biological networks and/or metabolic pathways related to T2D susceptibility genes, proteins or polypeptides.

Furthermore, a disclosed method or a test based on T2D susceptibility gene specific markers (e.g. polymorphic sites, expression or polypeptides) is useful in selecting drug therapy for patients with T2D.

The invention also discloses methods to assess the risk of an individual to develop T2D in mammals. A gene test recognizing the T2D risk allele homozygocity or carrier status of T2D susceptibility genes is useful in selecting prophylactic treatment for individuals having a high risk of T2D.

Yet in another embodiment of the invention, a test or a method based on T2D susceptibility gene specific markers (e.g. polymorphic sites, expression or polypeptides) is useful in selecting subjects testing treatments for T2D.

A test or a method of this invention based on T2D susceptibility gene specific markers (e.g. polymorphic sites, expression or polypeptides) is useful in selecting drug therapy for patients who might be at increased risk for adverse effects of drugs affecting T2D susceptibility gene activity.

If the less frequent, i.e. the minor, assumable mutated allele in the T2D susceptibility gene is risk-reducing, and if said mutation is a gene function reducing mutation, one can deduce that the gene function and/or activity would increase the risk of T2D. On that basis, drugs and other therapies such as gene therapies that reduce or inhibit the function or activity of the T2D susceptibility gene or the encoded protein would reduce the risk of the said disease and could be used to both prevent and treat the said disease.

Pharmaceutical Compositions

The present invention also pertains to pharmaceutical compositions comprising agents described herein, particularly polynucleotides, polypeptides and any fractions, variants or derivatives of T2D susceptibility genes, and/or agents that alter (e.g., enhance or inhibit) expression of T2D risk gene or genes, or activity of one or more polypeptides encoded by T2D susceptibility gene or genes as described herein. For instance, an agent that alters expression of T2D risk genes, or activity of one or more polypeptides encoded by T2D susceptibility genes or a T2D susceptibility polypeptide binding agent, binding partner, fragment, fusion protein or prodrug thereof, or polynucleotides of the present invention, can be formulated with a physiologically acceptable carrier or excipient to prepare a pharmaceutical composition. The carrier and composition can be sterile. The formulation should suit the mode of administration.

In a preferred embodiment pharmaceutical compositions comprise agent or agents reversing, at least partially, T2D associated changes in biological networks and/or metabolic pathways related to the T2D associated genes of this invention.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of T2D, and should be decided according to the judgment of a practitioner and each patient's circumstances. Effective doses may be extrapolated from dose-response curves derived from in vitro or animal model test systems.

Representative Target Population

An individual at risk of T2D is an individual who has at least one risk factor, such as family history of T2D or obesity, history of gestational diabetes, previously identified glucose intolerance, obesity, hypertriglyceridemia, low HDL cholesterol, HT and elevated BP, lack of physical activity, and an at-risk allele or haplotype with one or several T2D risk SNP markers.

In another embodiment of the invention, an individual who is at risk of T2D is an individual who has at least one risk-increasing allele in a T2D risk gene, in which the presence of the polymorphism is indicative of a susceptibility to T2D. The term “gene,” as used herein, refers to an entirety containing all regulatory elements located both upstream and downstream as well as within of a polypeptide encoding sequence of a gene and entire transcribed region of a gene including 5′ and 3′ untranslated regions of mRNA and the entire polypeptide encoding sequence including all exon and intron sequences (also alternatively spliced exons and introns) of a gene.

Assessment for At-Risk Alleles and At-Risk Haplotypes

The genetic markers are particular “alleles” at “polymorphic sites” associated with T2D. A nucleotide position in genome at which more than one sequence is possible in a population, is referred to herein as a “polymorphic site”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene e.g. in NCBI databases (www.ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences.

Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a T2D susceptibility gene. For example, a nucleotide change resulting in a change in polypeptide sequence can alter the physiological properties of a polypeptide dramatically by resulting in altered activity, distribution and stability or otherwise affect on properties of a polypeptide.

Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of a T2D susceptibility gene.

A “haplotype,” as described herein, refers to any combination of genetic markers (“alleles”). A haplotype can comprise two or more alleles and the length of a genome region comprising a haplotype may vary from few hundred bases up to hundreds of kilobases. As it is recognized by those skilled in the art the same haplotype can be described differently by determining the haplotype defining alleles from different nucleic acid strands. E.g. the haplotype CAC defined by the SNP markers rs2400503 (C/T), rs10515605 (A/G), and rs7709159 (C/T) is the same as the haplotype GTG in which the SNP markers rs2400503 (C/T), rs10515605 (A/G), and rs7709159 (C/T) are determined from the complementary strand or haplotype GAC in which the SNP marker rs2400503 (C/T) is determined from the complementary strand. The haplotypes described herein are found more frequently in individuals with T2D risk than in individuals without T2D risk. Therefore, these haplotypes have predictive value for detecting T2D risk or a susceptibility to T2D in an individual. Therefore, detecting haplotypes can be accomplished by methods known in the art for detecting sequences at polymorphic sites.

It is understood that the T2D associated at-risk alleles and at-risk haplotypes described in this invention may be associated with other “polymorphic sites” located in T2D associated genes of this invention. These other T2D associated polymorphic sites may be either equally useful as genetic markers or even more useful as causative variations explaining the observed association of at-risk alleles and at-risk haplotypes of this invention to T2D.

In certain methods described herein, an individual who is at risk for T2D is an individual in whom an at-risk allele or an at-risk haplotype is identified. In one embodiment, the at-risk allele or the at-risk haplotype is one that confers a significant risk of death. In one embodiment, significance associated with an allele or a haplotype is measured by an odds ratio. In a further embodiment, the significance is measured by a percentage. In one embodiment, a significant risk is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%,40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors, including the specific disease, the allele or the haplotype, and often, environmental factors.

An at-risk haplotype in, or comprising portions of, the T2D risk gene, is one where the haplotype is more frequently present in an individual at risk for T2D (affected), compared to the frequency of its presence in a healthy individual (control), and wherein the presence of the haplotype is indicative of T2D risk or susceptibility to T2D.

In a preferred embodiment, the method comprises assessing in an individual the presence or frequency of SNP markers in, comprising portions of, a T2D risk gene, wherein an excess or higher frequency of the SNP markers compared to a healthy control individual is indicative that the individual has T2D risk, or is susceptible to T2D. The presence of the haplotype is indicative of T2D risk, or a susceptibility to T2D, and therefore is indicative of an individual who falls within a target population for the treatment methods described herein.

Primers, Probes and Nucleic Acid Molecules

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Such probes and primers include polypeptide nucleic acids (Nielsen P E et al, 1991).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence.

In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

Antisense nucleic acid molecules of the invention can be designed using the nucleotide sequences of T2D risk genes and/or their complementary sequences and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule encoding a T2D risk gene, a fragment or a variant thereof has been cloned in antisense orientation (i.e., RNA transcribed from the expression vector will be complementary to the transcribed RNA of a T2D risk gene of interest).

The nucleic acid sequences of the T2D associated genes described in this invention can also be used to compare with endogenous DNA sequences in patients to identify genetic disorders (e.g., a predisposition for or susceptibility to T2D), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Polyclonal and Monoclonal Antibodies

The invention comprises polyclonal and monoclonal antibodies that bind to polypeptides of the invention. The term “antibody” as used herein refers to immunoglobulin molecules and immunologically active portions of immunoglobulin molecules, i.e., molecules that contain a binding site that specifically binds to an epitope (antigen, antigenic determinant). An antibody molecule that specifically binds to a polypeptide of the invention is a molecule that binds to an epitope present in said polypeptide or a fragment thereof, but does not substantially bind other molecules in a sample, e.g., a biological sample, which naturally contains the polypeptide. Examples of immunologically active portions of immunoglobulin molecules include F(ab) and F(ab′).sub.2 fragments which can be generated by treating the antibody with an enzyme such as pepsin. Polyclonal and/or monoclonal antibodies that specifically bind one form of the gene product but not to the other form of the gene product are also provided. Antibodies are also provided, that bind a portion of either the variant or the reference gene product that contains the polymorphic site or sites. The term “monoclonal antibody” or “monoclonal antibody composition”, as used herein refers to a population of antibody molecules that are directed against a specific epitope and are produced either by a single clone of B cells or a single hybridoma cell line. A monoclonal antibody composition thus typically displays a single binding affinity for a particular polypeptide of the invention with which it immunoreacts.

Polyclonal antibodies can be prepared as known by those skilled in the art by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique (Kohler G and Milstein C, 1975), the human B cell hybridoma technique (Kozbor D et al, 1982), the EBV-hybridoma technique (Cole S P et al, 1984), or trioma techniques (Hering S et al, 1988). To produce a hybridoma an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (Bierer B et al, 2002). Moreover, the ordinarily skilled worker will appreciate that there are many variations of such methods that also would be useful. Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide (Hayashi N et al, 1995; Hay B N et al, 1992; Huse W D et al, 1989; Griffiths A D et al, 1993). Kits for generating and screening phage display libraries are commercially available.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. An antibody specific for a polypeptide of the invention can facilitate the purification of a native polypeptide of the invention from biological materials, as well as the purification of recombinant form of a polypeptide of the invention from cultured cells (culture media or cells). Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue such as blood as part of a test predicting the susceptibility to T2D or as part of a clinical testing procedure, e.g., to, for example, determine the efficacy of a given treatment regimen. Antibodies can be coupled to various enzymes, prosthetic groups, fluorescent materials, luminescent materials, bioluminescent materials, and radioactive materials to enhance detection. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include .sup.125I, 131I, 35S or 3H.

Highly purified antibodies (e.g. monoclonal humanized antibodies specific to a polypeptide encoded by a T2D associated gene of this invention) may be produced using GMP-compliant manufacturing processes well known in the art. These “pharmaceutical grade” antibodies can be used in novel therapies modulating activity and/or function of a polypeptide encoded by a T2D associated gene of this invention or modulating activity and/or function of a metabolic pathway related a T2D associated gene of this invention.

Diagnostic Assays

The markers, probes, primers and antibodies described herein can be used in methods and kits used for risk assessment, diagnosis or prognosis of T2D or a disease or condition associated with T2D in a subject.

In one embodiment of the invention, diagnosis of risk or susceptibility to T2D (or diagnosis of or susceptibility to a disease or condition associated with T2D), is made by detecting one or several of at-risk alleles or at-risk haplotypes or a combination of at-risk alleles and at-risk haplotypes described in this invention in the subject's nucleic acid as described herein.

In one embodiment of the invention, diagnosis of risk or susceptibility to T2D (or diagnosis of or susceptibility to a disease or condition associated with T2D), is made by detecting one or several of polymorphic sites which are associated with at-risk alleles or/and at-risk haplotypes described in this invention in the subject's nucleic acid. Diagnostically the most useful polymorphic sites are those altering the polypeptide structure of a T2D associated gene due to a frame shift; due to a premature stop codon, due to an amino acid change or due to abnormal mRNA splicing. Nucleotide changes resulting in a change in polypeptide sequence in many cases alter the physiological properties of a polypeptide by resulting in altered activity, distribution and stability or otherwise affect on properties of a polypeptide. Other diagnostically useful polymorphic sites are those affecting transcription of a T2D associated gene or translation of it's mRNA due to altered tissue specificity, due to altered transcription rate, due to altered response to physiological status, due to altered translation efficiency of the mRNA and due to altered stability of the mRNA. The presence of nucleotide sequence variants altering the polypeptide structure of T2D associated genes or altering the expression of T2D associated genes is diagnostic for susceptibility to T2D.

For diagnostic applications, there may be polymorphisms informative for prediction of disease risk, which are in linkage disequilibrium with the functional polymorphism. Such a functional polymorphism may alter splicing sites, affect the stability or transport of mRNA, or otherwise affect the transcription or translation of the nucleic acid. The presence of nucleotide sequence variants associated with a functional polymorphism is diagnostic for susceptibility to T2D. While we have genotyped and included a limited number of example SNP markers in the experimental section, any functional, regulatory or other mutation or alteration described above in any of the T2D risk genes identified herein is expected to predict the risk of T2D.

In diagnostic assays determination of the nucleotides present in one or several of the T2D associated SNP markers of this invention, as well as polymorphic sites associated with T2D associated SNP markers of this invention, in an individual's nucleic acid can be done by any method or technique which can accurately determine nucleotides present in a polymorphic site. Numerous suitable methods have been described in the art (see e.g. Kwok P-Y, 2001; Syvanen A-C, 2001), these methods include, but are not limited to, hybridization assays, ligation assays, primer extension assays, enzymatic cleavage assays, chemical cleavage assays and any combinations of these assays. The assays may or may not include PCR, solid phase step, a microarray, modified oligonucleotides, labeled probes or labeled nucleotides and the assay may be multiplex or singleplex. As it is obvious in the art the nucleotides present in a polymorphic site can be determined from either nucleic acid strand or from both strands.

In another embodiment of the invention, diagnosis of a susceptibility to T2D can be assessed by examining transcription of one or several T2D associated genes. Alterations in transcription can be assessed by a variety of methods described in the art, including e.g. hybridization methods, enzymatic cleavage assays, RT-PCR assays and microarrays. A test sample from an individual is collected and the alterations in the transcription of T2D associated genes are assessed from the RNA present in the sample. Altered transcription is diagnostic for a susceptibility to T2D.

In another embodiment of the invention, diagnosis of a susceptibility to T2D can also be made by examining expression and/or structure and/or function of a T2D susceptibility polypeptide. A test sample from an individual is assessed for the presence of an alteration in the expression and/or an alteration in structure and/or function of the polypeptide encoded by a T2D risk gene, or for the presence of a particular polypeptide variant (e.g., an isoform) encoded by a T2D risk gene. An alteration in expression of a polypeptide encoded by a T2D risk gene can be, for example, quantitative (an alteration in the quantity of the expressed polypeptide, i.e., the amount of polypeptide produced) or qualitative (an alteration in the structure and/or function of a polypeptide encoded by a T2D risk gene, i.e. expression of a mutant T2D susceptibility polypeptide or of a different splicing variant or isoform). In a preferred embodiment, detecting a particular splicing variant encoded by a T2D risk gene or a particular pattern of splicing variants makes diagnosis of the disease or condition associated with T2D or a susceptibility to a disease or condition associated with T2D.

Alterations in expression and/or structure and/or function of a T2D susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the T2D susceptibility polypeptide or it's fragment or from substrates and reaction products of said polypeptide. A control sample is a sample that corresponds to the test sample (e.g., is from the same type of cells), and is from an individual who is not affected by T2D. An alteration in the expression or composition of a polypeptide encoded by a T2D susceptibility gene of the invention in the test sample, as compared with the control sample, is indicative of a susceptibility to T2D.

Western blotting analysis, using an antibody as described above that specifically binds to a polypeptide encoded by a mutant T2D risk gene or an antibody that specifically binds to a polypeptide encoded by a non-mutant gene, or an antibody that specifically binds to a particular splicing variant encoded by a T2D risk gene can be used to identify the presence or absence in a test sample of a particular polypeptide encoded by a polymorphic or mutant T2D risk gene. The presence of a polypeptide encoded by a polymorphic or mutant gene, or the absence of a polypeptide encoded by a non-polymorphic or non-mutant gene, is diagnostic for a susceptibility to T2D, as is the presence (or absence) of particular splicing variants encoded by a T2D risk gene.

In one embodiment of this method, the level or amount of a polypeptide encoded by a T2D risk gene in a test sample is compared with the level or amount of the same polypeptide encoded by the same T2D risk gene in a control sample. A level or amount of the polypeptide in the test sample that is higher or lower than the level or amount of the polypeptide in the control sample, such that the difference is statistically significant, is indicative of an alteration in the expression of the polypeptide encoded by a T2D risk gene, and is diagnostic for a susceptibility to T2D. Alternatively, the composition of the polypeptide encoded by a T2D risk gene in a test sample is compared with the composition of the polypeptide encoded by a T2D risk gene in a control sample (e.g., the presence of different splicing variants). A difference in the composition of the polypeptide in the test sample, as compared with the composition of the polypeptide in the control sample, is diagnostic for a susceptibility to T2D. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to T2D.

In another embodiment, assessment of the splicing variant or isoform(s) of a polypeptide encoded by a polymorphic or mutant T2D risk gene can be performed. The assessment can be performed directly (e.g., by examining the polypeptide itself), or indirectly (e.g., by examining the mRNA encoding the polypeptide, such as through mRNA profiling). For example, probes or primers as described herein can be used to determine which splicing variants or isoforms are encoded by a T2D risk gene mRNA, using standard methods.

The presence in a test sample of a particular splicing variant(s) or isoform(s) associated with T2D or risk of T2D, or the absence in a test sample of a particular splicing variant(s) or isoform(s) not associated with T2D or risk of T2D, is diagnostic for a disease or condition associated with a T2D risk gene or a susceptibility to a disease or condition associated with a T2D risk gene. Similarly, the absence in a test sample of a particular splicing variant(s) or isoform(s) associated with T2D or risk of T2D, or the presence in a test sample of a particular splicing variant(s) or isoform(s) not associated with T2D or risk of T2D, is diagnostic for the absence of disease or condition associated with a T2D risk gene or a susceptibility to a disease or condition associated with a T2D risk gene.

The invention further pertains to a method for the diagnosis and identification of susceptibility to T2D in an individual, by assessing markers present in at-risk alleles or at-risk haplotypes of T2D risk genes. In one embodiment, the at-risk allele or the at-risk haplotype is an allele or a haplotype for which the presence of the allele or the haplotype increases the risk of T2D significantly. Although it is to be understood that identifying whether a risk is significant may depend on a variety of factors, including the specific disease, the haplotype, and often, environmental factors, the significance may be measured by an odds ratio or a percentage. In a further embodiment, the significance is measured by a percentage. In one embodiment, a significant risk is measured as an odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, an odds ratio of at least 1.2 is significant. In a further embodiment, an odds ratio of at least about 1.5 is significant. In a further embodiment, a significant increase or decrease in risk is at least about 1.7. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase or reduction in risk is at least about 50%. It is understood however, that identifying whether a risk is medically significant may also depend on a variety of factors, including the specific disease, the allele or the haplotype, and often, environmental factors.

The invention also pertains to methods of diagnosing risk or a susceptibility to T2D in an individual, comprising screening for an at-risk haplotype in a T2D risk gene that is more frequently present in an individual susceptible to T2D (affected), compared to the frequency of its presence in a healthy individual (control), wherein the presence of the haplotype is indicative of risk or susceptibility to T2D.

Yet in another embodiment, a susceptibility to T2D can be diagnosed by assessing the status and/or function of biological networks and/or metabolic pathways related to one or several polypeptides encoded by T2D risk genes of this invention. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop a T2D is evaluated by comparing observed status and/or function of biological networks and or metabolic pathways of a subject to the status and/or function of biological networks and or metabolic pathways of healthy controls.

Kits (e.g., reagent kits) useful in the methods of diagnosis comprise components useful in any of the methods described herein, including for example, PCR primers, hybridization probes or primers as described herein (e.g., labeled probes or primers), reagents for genotyping SNP markers, reagents for detection of labeled molecules, restriction enzymes (e.g., for RFLP analysis), allele-specific oligonucleotides, DNA polymerases, RNA polymerases, marker enzymes, antibodies which bind to altered or to non-altered (native) T2D susceptibility polypeptide, means for amplification of nucleic acids comprising one or several T2D risk genes, or means for analyzing the nucleic acid sequence of one or several T2D risk genes or for analyzing the amino acid sequence of one or several T2D susceptibility polypeptides, etc. In one embodiment, a kit for diagnosing susceptibility to T2Dcan comprise primers for nucleic acid amplification of fragments from a T2D risk gene comprising markers defining an at-risk haplotype that is more frequently present in an individual susceptible to T2D. The primers can be designed using portions of the nucleic acid sequence flanking SNPs that are indicative of T2D.

This invention is based on the principle that one or a small number of genotype analyses are performed, and the mutations to be typed are selected on the basis of their ability to predict T2D. For this reason any method to genotype mutations in a genomic DNA sample can be used. If non-parallel methods such as real-time PCR are used, the genotype analyses are done in a row. The PCR reactions may be multiplexed or carried out separately in a row or in parallel aliquots.

Thus, the detection method of the invention may further comprise a step of combining information concerning age, gender, smoking status, physical activity, waist-to-hip circumference ratio (cm/cm), the subject family history of T2D or obesity, history of gestational diabetes, previously identified glucose intolerance, obesity, hypertriglyceridemia, low HDL cholesterol, HT and elevated BP. The detection method of the invention may also further comprise a step determining blood, serum or plasma glucose, total cholesterol, HDL cholesterol, LDL cholesterol, triglyceride, apolipoprotein B and Al, fibrinogen, ferritin, transferrin receptor, C-reactive protein, serum or plasma insulin concentration.

The score that predicts the probability of T2D may be calculated e.g. using a multivariate failure time model or a logistic regression equation. The results from the further steps of the method as described above render possible a step of calculating the probability of T2D using a logistic regression equation as follows.

Probability of T2D=1/[1+e(−(−a+Σ(bi*Xi))], where e is Napier's constant, Xi are variables related to the T2D, bi are coefficients of these variables in the logistic function, and a is the constant term in the logistic function, and wherein a and bi are preferably determined in the population in which the method is to be used, and Xi are preferably selected among the variables that have been measured in the population in which the method is to be used. Preferable values for bᵢ are between −20 and 20; and for i between 0 (none) and 100,000. A negative coefficient bi implies that the marker is risk-reducing and a positive that the marker is risk-increasing. Xi are binary variables that can have values or are coded as 0 (zero) or 1 (one) such as SNP markers. The model may additionally include any interaction (product) or terms of any variables Xi, e.g. biXi. An algorithm is developed for combining the information to yield a simple prediction of T2D as percentage of risk in one year, two years, five years, 10 years or 20 years. Alternative statistical models are failure-time models such as the Cox's proportional hazards' model, other iterative models and neural networking models.

The test can be applied to test the risk of developing a T2D in both healthy persons, as a screening or predisposition test and high-risk persons (who have e.g. family history of T2D or history of gestational diabetes or previous glucose intolerance or obesity or any combination of these or elevated level of any other T2D risk factor).

The method can be used in the prediction and early diagnosis of T2D in adult persons, stratification and selection of subjects in clinical trials, stratification and selection of persons for intensified preventive and curative interventions. The aim is to reduce the cost of clinical drug trials and health care.

Monitoring Progress of Treatment

The current invention also pertains to methods of monitoring the effectiveness of a treatment of T2D based on the expression (e.g., relative or absolute expression) of one or more T2D risk genes. The T2D risk susceptibility gene mRNA, or polypeptide it is encoding or biological activity of the encoded polypeptide can be measured in a tissue sample (e.g. peripheral blood sample or adipose tissue biopsy). An assessment of the levels of expression or biological activity of the polypeptide can be made before and during treatment with T2D therapeutic agents.

Alternatively the effectiveness of a treatment of T2D can be followed by assessing the status and/or function of biological networks and/or metabolic pathways related to one or several polypeptides encoded by T2D risk genes of this invention. Status and/or function of a biological network and/or a metabolic pathway can be assessed e.g. by measuring amount or composition of one or several polypeptides, belonging to the biological network and/or to the metabolic pathway, from a biological sample taken from a subject before and during a treatment. Alternatively status and/or function of a biological network and/or a metabolic pathway can be assessed by measuring one or several metabolites belonging to the biological network and/or to the metabolic pathway, from a biological sample before and during a treatment. Effectiveness of a treatment is evaluated by comparing observed changes in status and/or function of biological networks and or metabolic pathways following treatment with T2D therapeutic agents to the data available from healthy subjects.

For example, in one embodiment of the invention, an individual who is a member of the target population can be assessed for response to treatment with an T2D inhibitor, by examining T2D risk gene encoding polypeptide biological activity or absolute and/or relative levels of T2D risk gene encoding polypeptide or mRNA in peripheral blood in general or in specific cell fractions or in a combination of cell fractions.

In addition, variations such as SNP markers defining haplotypes or mutations within or near (e.g. within 1 to 200 kb) of the T2D risk gene may be used to identify individuals who are at higher risk for T2D to increase the power and efficiency of clinical trials for pharmaceutical agents to prevent or treat T2D or their complications. The presence of at-risk haplotypes and other variations may be used to exclude or fractionate patients in a clinical trial who are likely to have involvement of another pathway in their T2D in order to enrich patients who have pathways involved that are relevant regarding to the treatment tested and boost the power and sensitivity of the clinical trial. Such variations may be used as a pharmacogenetic test to guide the selection of pharmaceutical agents for individuals.

This application includes sequence listing and tables that are submitted in electronic form. The sequence listing and tables are submitted herewith on one original and one duplicate compact disc (in compliance with 37 C.F.R. § 1.52(e)) designated respectively as Copy 1 and Copy 2, and labeled in compliance with 37 C.F.R. § 1.52(e)(6). All the material in the sequence listing and tables on compact disc is hereby incorporated in their entirety herein by reference, and identified by the following data of file names, creation date and size in bytes:

| FILE NAME | CREATED | SIZE IN BYTES |
|---|---|---|
| Sequence listing.txt | 05-Dec-05 | 684 000 |
| Table1_T2D.txt | 05-Dec-05 | 144 000 |
| Table2_T2D.txt | 14-Nov-05 | 76 700 |
| Table3_T2D.txt | 14-Nov-05 | 100 000 |
| Table4_T2D.txt | 14-Nov-05 | 10 300 |
| Table5_T2D.txt | 14-Nov-05 | 115 000 |
| Table6_T2D.txt | 14-Nov-05 | 116 000 |
| Table7_T2D.txt | 14-Nov-05 | 26 300 |
| Table8_T2D.txt | 09-Dec-05 | 86 200 |
| Table9_T2D.txt | 09-Dec-05 | 83 300 |
| Table10_T2D.txt | 09-Dec-05 | 69 500 |
| Table11_T2D.txt | 12-Dec-05 | 3 050 |
| Table12_T2D.txt | 12-Dec-05 | 3 130 |
| Table13_T2D.txt | 20-Dec-05 | 53 900 |

Experimental Section

Study Populations: The KIHD and NSP Cohorts

KIHD: The Kuopio Ischaemic Heart Disease Risk Factor Study (KIHD) is an ongoing prospective population-based cohort study, which was designed to investigate genetic and other risk factors for cardiovascular and metabolic diseases and related outcomes in the East Finland founder population, known for its genetic homogeneity and high occurrence of CHD (Salonen J T 1988, Salonen J T et al 1998, 1999, Tuomainen T-P et al 1999).

The study population was a random age-stratified sample of men living in Eastern Finland who were 42, 48, 54 or 60 years old at baseline examinations in 1984-1989. A total of 2682 men were examined during 1984-89. The male cohort was complemented by a random population sample of 920 women, first examined during 1998-2001, at the time of the 11-year follow up of the male cohort. The recruitment and examination of the subjects has been described previously in detail (Salonen J T, 1988). The University of Kuopio and University Hospital Ethics Committee approved the study. All participants gave their written informed consent.

NSP: In addition to the existing KIHD cohort, a large population cohort was examined in 2003 in Eastern Finland. This “North Savo Project” (NSP) includes the collection of disease, family, drug response and contact information. By October 2004, 17,100 participants have been surveyed. The study consists of the identification of probands with T2D, the collection of blood for DNA extraction and consent to use it. In the second phase, patients with T2D and T2D-free controls were examined.

Definition of Cases and Controls

The subjects of the present study were participants in either the KIHD or the NSP. In the KIHD, fasting blood glucose was measured using a glucose dehydrogenase method after precipitation of proteins by trichloroacetic acid. Serum insulin was determined with a Novo Biolabs radioimmunoassay kit (Novo Nordisk). In the NSP, prevalent diabetes was assessed by medication review and fasting blood glucose level, obtained from whole blood samples after at least 12 hours of overnight fasting and measured with the glucose dehydrogenase method after precipitation of the proteins with trichloroacetic acid (Granutest 100, Merck). A person was considered diabetic if he/she currently used diet or took medication to control blood glucose or if he/she had a fasting blood glucose level of >6.7 mmol/L (120 mg/dL).

The cases had T2D and family history of T2D. All T2D cases (probands) had at least one additional affected relative, who was parent, sibling or offspring of the proband. Most of them had more than one additional affected family member. The controls had neither T2D nor family history of T2D. During the first phase of the study 93 cases and 157 controls were analyzed (first analysis) using the technology access version or commercial version of Affymetrix GeneChip® Human Mapping 100 k assay. In the second phase of the study 30 cases and 29 controls were added, so that a second analysis was based on a set of 123 and 186 controls using either the technology access version or a commercial version of Affymetrix GeneChip® Human Mapping 100 k assay. The third analysis was based on 80 cases and 30 controls (who were included in the second analysis) using only the commercial version of Affymetrix GeneChip® Human Mapping 100 k assay. Combined results from the analyses are presented in the results section.

Other Phenotypic Measurements

Age and tobacco smoking were recorded on a self-administered questionnaire checked by an interviewer. HDL fractions were separated from fresh serum by combined ultracentrifugation and precipitation. The cholesterol contents of lipoprotein fractions and serum triglycerides were measured enzymatically. Both systolic and diastolic BPs were measured in the morning by a nurse with a random-zero mercury sphygmomanometer. The measuring protocol included three measurements in supine, one in standing and two in sitting position with 5-minutes intervals. The mean of all six measurements were used as SBP and DBP (Salonen J T et al, 1998). Body mass index (BMI) was computed as the ratio of weight to the square of height (kg/m 2). Waist-to-hip ratio (WHR) was calculated as the ratio of waist circumference (average of one measure taken after inspiration and one taken after expiration at the midpoint between the lowest rib and the iliac crest) to hip circumference (measured at the level of the trochanter major).

Genomic DNA Isolation and Quality Testing

High molecular weight genomic DNA samples were extracted from frozen venous whole blood using standard methods and dissolved in standard TE buffer. The quantity and purity of each DNA sample was evaluated by measuring the absorbance at 260 and 280 nm and integrity of isolated DNA samples was evaluated with 0.9% agarose gel electrophoresis and Ethidiumbromide staining. A sample was qualified for genome wide scan (GWS) analysis if A260/A280 ratio was ≧1.7 and/or average size of isolated DNA was over 20 kb in agarose gel electrophoresis. Before GWS analysis samples were diluted to concentration of 50 ng/μl in reduced EDTA TE buffer (TEKnova).

Genome-Wide Scan

Genotyping of SNP markers was performed by using either the technology access version or a commercial version of Affymetrix GeneChip® Human Mapping 100 k assay. The assay consisted of two arrays, Xba and Hind, which were used to genotype over 100,000 SNP markers from each DNA sample. The assays were performed according to the instructions provided by the manufacturer. A total of 250 ng of genomic DNA was used for each individual assay. DNA sample was digested with either Xba I or Hind III enzyme (New England Biolabs, NEB) in the mixture of NE Buffer 2 (1×; NEB), bovine serum albumin (1×; NEB), and either Xba I or Hind III (0.5 U/μl; NEB) for 2 h at +37° C. followed by enzyme inactivation for 20 min at +70° C. Xba I or Hind III adapters were then ligated to the digested DNA samples by adding Xba or Hind III adapter (0.25 μM, Affymetrix), T4 DNA ligase buffer (1×; NEB), and T4 DNA ligase (250 U; NEB). Ligation reactions were allowed to proceed for 2 h at +16° C. followed by 20 min incubation at +70° C. Each ligated DNA sample was diluted with 75 μl of molecular biology-grade water (BioWhittaker Molecular Applications/Cambrex).

Diluted ligated DNA samples were subjected to three to four identical 100 μl volume polymerase chain reactions (PCR) by implementing a 10 μl aliquot of DNA sample with Pfx Amplification Buffer (1×; Invitrogen), PCR Enhancer (1×; Invitrogen), MgSO₄ (1 mM; Invitrogen), dNTP (300 μM each; Takara), PCR primer (1 μM; Affymetrix), and Pfx Polymerase (0.05 U/el; Invitrogen). The PCR was allowed to proceed for 3 min at +94° C, followed by 30 cycles of 15 sec at +94° C., 30 sec at +60° C., 60 sec at +68° C., and finally for the final extension for 7 min at +68° C. The performance of the PCR was checked by standard 2% agarose gel electrophoresis in 1×TBE buffer for 1 h at 120V.

PCR products were purified according to Affymetrix manual using MinElute 96 UF PCR Purification kit (Qiagen) by combining all three to four PCR products of an individual sample into same purification reaction. The purified PCR products were eluted with 40 μl of EB buffer (Qiagen), and the yields of the products were measured at the absorbance 260 nm. A total of 40 μg of each PCR product was then subjected to fragmentation reaction consisting of 0.2 U of fragmentation reagent (Affymetrix) in 1× Fragmentation Buffer. Fragmentation reaction was allowed to proceed for 35 min at +37° C. followed by 15 min incubation at +95° C. for enzyme inactivation. Completeness of fragmentation was checked by running an aliquot of each fragmented PCR product in 4% TBE gel (4% NuSieve 3:1 Plus Agarose; BMA Reliant precast) for 30-45 min at 120V.

Fragmented PCR products were then labelled using 1× Terminal Deoxinucleotidyl Transferase (TdT) buffer (Affymetrix), GeneChip DNA Labeling Reagent (0.214 mM; Affymetrix), and TdT (1.5 U/μl; Affymetrix) for 2 h at +37° C. followed by 15 min at +95° C. Labeled DNA samples were combined with hybridization buffer consisting of 0.056 M MES solution (Sigma), 5% DMSO (Sigma), 2.5× Denhardt's solution (Sigma), 5.77 mM EDTA (Ambion), 0.115 mg/ml Herring Sperm DNA (Promega), 1× Oligonucleotide Control reagent (Affymetrix), 11.5 μg/ml Human Cot-1 (Invitrogen), 0.0115% Tween-20 (Pierce), and 2.69 M Tetramethyl Ammonium Chloride (Sigma). DNA-hybridization buffer mix was denatured for 10 min at +95° C., cooled on ice for 10 sec and incubated for 2 min at +48° C. prior to hybridization onto corresponding Xba or Hind GeneChip(V array. Hybridization was completed at +48° C. for 16-18 h at 60 rpm in an Affymetrix GeneChip Hybridization Oven. Following hybridization, the arrays were stained and washed in GeneChip Fluidics Station 450 according to fluidics station protocol Mapping10Kv1_{—}450 (technology access version arrays) or Mapping100Kv1_{—}450 (commercial version arrays) as recommended by the manufacturer. Arrays were scanned with GeneChip 3000 Scanner, and the genotype calls for each of the SNP markers on the array were generated using Affymetrix Genotyping Tools (GTT) software (technology access version arrays, confidence score in SNP calling algorithm was adjusted to 0.20) or using GeneChip DNA Analysis Software 2.0 (GDAS) (commercial version arrays) with standard settings provided by the manufacturer.

Initial SNP Selection for Statistical Analysis

Prior to the statistical analysis, SNP quality was assessed on the basis of three values: the call rate (CR), minor allele frequency (MAF), and Hardy-Weinberg equilibrium (H-W). The CR is the proportion of samples genotyped successfully. It does not take into account whether the genotypes are correct or not. The call rate was calculated as: CR=number of samples with successful genotype call/total number of samples. The MAF is the frequency of the allele that is less frequent in the study sample. MAF was calculated as: MAF=min(p, q), where p is frequency of the SNP allele ‘A’ and q is frequency of the SNP allele ‘B’; p=(number of samples with “AA”-genotype+0.5*number of samples with “AB”-genotype)/total number of samples with successful genotype call; q=1−p. SNPs that are homozygous (MAF=0) cannot be used in genetic analysis and were thus discarded. H-W equilibrium is tested for controls. The test is based on the standard Chi-square test of goodness of fit. The observed genotype distribution is compared with the expected genotype distribution under H-W equilibrium. For two alleles this distribution is p2, 2pq, and q2 for genotypes ‘AA’, ‘AB’ and ‘BB’, respectively. If the SNP is not in H-W equilibrium it can be due to genotyping error or some unknown population dynamics (e.g. random drift, selection).

Based on the control group, only the SNPs that had CR>50%, MAF>1%, and were in H-W equilibrium (Chi-square test statistic <23.93) were used in the statistical analysis. A total of 100,848 SNPs fulfilled the above criteria and were included in the statistical analysis.

Statistical Methods

Single SNP Analysis

Differences in allele distributions between cases and controls were screened for all SNPs. The screening was carried out using the standard Chi-square independence test with 1 df (allele distribution, 2×2 table). SNPs that gave a P-value less than 0.005 (Chi-square distribution with 1 df of 7.88 or more) were considered as statistically significant and reported in the table 1. Odds ratio was calculated as ad/bc, where a is the number of minor alleles in cases, b is the number of major alleles in cases, c is the number of minor allele in controls, and d is the number of major alleles in controls. Minor allele was defined as the allele for a given SNP that has smaller frequency than the other allele in the control group.

Haplotype Analysis

The data set was analyzed with a haplotype pattern mining algorithm with HPM software (Toivonen HT et al, 2000). For HPM software, genotypes must be phase known to determine which alleles come from the mother and which from the father. Without family data, phases must be estimated based on population data. We used HaploRec-program (Eronen L et al, 2004) to estimate the phases. HPM is very fast and can handle a large number of SNPs in a single run

For phase-known data HPM finds all haplotype patterns that are in concordance with the phase configuration. The length of the haplotype patterns can vary. As an example, if there are four SNPs and an individual has alleles A T for SNP1, C C for SNP2, C G for SNP3, and A C for SNP4, then HPM considers haplotype patterns that are in concordance with the estimated phase (done by HaploRec). If the estimated phase is ACGA (from the mother/father) and TCCC (from the father/mother) then HPM considers only two patterns (of length 4 SNPs): ACGA and TCCC.

A SNP is scored based on the number of times it is included in a haplotype pattern that differs between cases and controls (a threshold Chi-square value can be selected by the user). Significance of the score values is tested based on permutation tests.

Several parameters can be modified in the HPM program including the Chi-square threshold value (−x), the maximum haplotype pattern length (−l), the maximum number of wildcards that can be included in a haplotype pattern (−w), and the number of permutation tests in order to estimate the P-value (−p). Wildcards allow gaps in haplotypes. HaploRec+ HPM was run with the following parameter settings: haplotype analysis with 5 SNPs (−x9−l5−w1−p1000) and haplotype analysis with 8 SNPs (−x9−l8−w1−p10000). HaploRec+HPM was based on the order of the SNP given in dbSNP124. Based on 10,000 replicates (−p10000) SNPs that gave a P-value less than 0.005 were considered as statistically significant and reported in tables 2-7.

Definition of Terms Used in the Haplotype Analysis Results

The term “haplotype genomic region” or “haplotype region” refers to a genomic region that has been found significant in the haplotype analysis (HPM or similar statistical method/program). The haplotype region is defined as 100 Kbp up/downstream from the physical position of the first/last SNP that was included in the statistical analysis (haplotype analysis) and was found statistically significant. This region is given in base pairs based on the given genome build e.g. SNP physical position (base pair position) according to NCBI Human Genome Build 35.

The term “haplotype” as described herein, refers to any combination of alleles e.g. C A G G that is found in the given genetic markers e.g rs1418754, rs2797573, rs787663, rs10509650. A defined haplotype gives the name of the genetic markers (dbSNP rs-id for the SNPs) and the alleles. As it is recognized by those skilled in the art, the same haplotype can be described differently by determining alleles from different strands e.g. the haplotype rs1418754, rs2797573, rs787663, rs10509650 (C A G G) is the same as haplotype rs1418754, rs2797573, rs787663, rs10509650 (G T C C) in which the alleles are determined from the other strand, or haplotype rs1418754, rs2797573, rs787663, rs10509650 (G A G G), in which the first allele is determined from the other strand.

The haplotypes described herein, e.g. having markers such as those shown in tables 2-7, are found more frequently in individuals with T2D than in individuals without T2D. Therefore, these haplotypes have predictive value for detecting T2D or a susceptibility to T2D in an individual. Therefore, detecting haplotypes can be accomplished by methods known in the art for detecting sequences at polymorphic sites.

It is understood that the T2D associated at-risk alleles and at-risk haplotypes described in this invention may be associated with other “polymorphic sites” located in T2D associated genes of this invention. These other T2D associated polymorphic sites may be either equally useful as genetic markers or even more useful as causative variations explaining the observed association of at-risk alleles and at-risk haplotypes of this invention to T2D.

Results

In table 1 are summarized the characteristics of the SNP markers with the strongest association with T2D in the individual marker analysis combined from both the first and second genome scans. SNP identification number according to NCBI dbSNP database build 124. SNP physical position according to NCBI Human Genome Build 35.1. Gene locus as, reported by NCBI dbSNP database build 124. SNP flanking sequence provided by Affymetrix “csv” commercial Human Mapping 100K array annotation files. The genes positioned within 100 Kbp up/downstream from the physical position of the SNPs are provided based on the NCBI Human Genome Build 35.1.

In table 2 are summarized the characteristics of the haplotype genomic regions with the strongest association with T2D in the haplotype sharing analysis with 5 SNPs (HaploRec+HPM) from the analysis of the first genome scan. SNP identification number according to NCBI dbSNP database build 124. SNP physical position according to NCBI Human Genome Build 35.1. Haplotype gene content, genes positioned within 100 Kbp up/downstream from the physical position of the SNPs bordering the haplotype genomic region found using NCBI MapViewer, based on NCBI Human Genome Build 35.1. SNP flanking sequence provided by Affymetrix “csv” commercial Human Mapping 100K array annotation files.

In table 3 are summarized the characteristics of the haplotype genomic regions with the strongest association with T2D in the haplotype sharing analysis with 8 SNPs (HaploRec+HPM) from the analysis of the first genome scan. Annotation information as in table 2.

In table 4 are listed haplotype block regions and corresponding at-risk haplotypes with the strongest association with T2D based on the Chi-square value from the first genome scan. The Chi-square value is calculated using the 2×2 Chi-square test with the cells containing the number of individuals 1) carrying the at-risk haplotype and having T2D, 2) carrying the at-risk haplotype and not having T2D, 3) not carrying the at-risk haplotype and having T2D, and 4) not carrying the at-risk haplotype and not having T2D. SNP identification number according to NCBI dbSNP database build 124.

In table 5 are summarized the characteristics of the haplotype genomic regions with the strongest association with T2D in the haplotype sharing analysis with 5 SNPs (HaploRec+HPM) from the analysis of the second genome scan. Annotation information as in table 2.

In table 6 are summarized the characteristics of the haplotype genomic regions with the strongest association with T2D in the haplotype sharing analysis with 8 SNPs (HaploRec+HPM) from the analysis of the second genome scan. Annotation information as in table 2.

In table 7 are listed haplotype block regions and corresponding at-risk haplotypes with the strongest association with T2D based on the Chi-square value from the second genome scan. The Chi-square value is calculated as for table 4.

In table 8 are summarized the characteristics of the SNP markers with the strongest association with T2D in the individual marker analysis from both the third genome scan. Annotation information as in table 1.

In table 9 are summarized the characteristics of the haplotype genomic regions with the strongest association with T2D in the haplotype sharing analysis with 5 SNPs (HaploRec+HPM) from the analysis of the third genome scan. Annotation information as in table 2.

In table 10 are summarized the characteristics of the haplotype genomic regions with the strongest association with T2D in the haplotype sharing analysis with 8 SNPs (HaploRec+HPM) from the analysis of the third genome scan. Annotation information as in table 2.

In table 11 and 12 are presented results of a multivariate logistic model predicting T2D. The model is adjusted for the type of microarray used i.e. whether the samples were analyzed with the early access Affymetrix 100K or with the commercial version of the Affymetrix 100K array, by including a dummy (0, 1) variable. The risk allele was considered as independet variable in the regression model. The coding of SNPs: 0, 1, 2, where code 2 denotes homozygocity of the minor allele, code 1 heterozygocity and code 0 homozygocity of non-minor allele. Annotation information as in previous tables.

In table 13 are listed all genes found associated with T2D according to point wise and haplotype analyses in all three genome scans. Gene name according to HUGO Gene Nomenclature Committee (HGNC).

Implications and Conclusions

We have found 4685 SNP markers associated with T2D in a population-based set of familial cases and healthy controls without family history. Of these, 1637 were identified in the analysis of individual SNPs and 3751 in the haplotype sharing analysis. Of the 4685 markers, 703 predicted T2D in both types of statistical analysis. Of the 4685 SNP markers associated with the risk of T2D 2407 SNP markers were intragenic.

The results of the point wise and haplotype analyses identified a total of 3048 genes associated with T2D, of which 903 genes had at least one of the 4685 SNP markers physically linked to the gene.

Thus, we have discovered a total of 3048 T2D genes, in which any genetic marker can be used to predict T2D, and thus these markers can be used as part of molecular diagnostic tests of T2D predisposition. In addition, we have disclosed a set of 4685 SNP markers which are predictive of T2D. The markers can also be used as part of pharmacogenetic tests which predict the efficacy and adverse reactions of antihyperglicemic agents and compounds. The genes discovered are also targets to new therapies of T2D, such as drugs. Other therapies are molecular, including gene transfer. The new genes can also be used to develop and produce new transgenic animals for studies of antihypertensive agents and compounds.

While this invention has been particularly shown and described with reference to preferred embodiments thereof, it will be understood by those skilled in the art that various changes in form and details may be made therein without departing from the spirit and scope of the invention as defined by the appended claims.

### REFERENCES

Altshuler D, Hirschhorn J N, Klannemark M, Lindgren C M, Vohl M C, et al. 2000. The common PPARgamma Pro12Ala polymorphism is associated with decreased risk of type 2 diabetes. Nat. Genet. 26:76-80.
American Diabetes Association. 2003. Report of the Expert Committee on the Diagnosis and Classification of Diabetes Mellitus. Diabetes Care 26:S5-S20.
Barnett A H, Eff C, Leslie R D, Pyke D A. 1981. Diabetes in identical twins. A study of 200 pairs. Diabetologia 20:87-93.
Bell G I, Xiang K, Newman M V, Wu S, Wright L G, Fajans S S, Spielman R S, Cox N J. 1991. Gene for non-insulin-dependent diabetes mellitus (maturity-onset diabetes of the young subtype) is linked to DNA polymorphism on human chromosome 20q. Proc Natl Acad Sci 88:1484-1488.
Berg A H, Combs T P, Scherer P E. 2002. ACRP30/adiponectin: an adipokine regulating glucose and lipid metabolism. Trends Endocrinol Metab. 13:84-9.
Bierer B, Coligan J E, Margulies D H, Shevach E M, Strober W. 2002. Current Protocols in Immunology. NY:John Wiley & Sons.
Boden G. 2001. Free fatty acids-the link between obesity and insulin resistance. Endocr Pract. 7:44-51.
Buchanan T A.2003. Pancreatic beta-cell loss and preservation in type 2 diabetes. Clin Ther. 25 Suppl B:B32-46.
Byrne M M, Sturis J, Menzel S, Yamagata K, Fajans S S, Dronsfield M J, Bain S C, Hattersley A T, Velho G, Froguel P, Bell G I, Polonsky K S. 1996. Altered insulin secretory response to glucose in diabetic and nondiabetic subjects with mutations in the diabetes susceptibility gene MODY3 on chromosome 12. Diabetes 45:1503-1510.
Chiu K C, Province M A, Dowse G K, Zimmet P Z, Wagner G, et al. 1992. A genetic marker at the glucokinase gene locus for type 2 (non-insulin-dependent) diabetes mellitus in Mauritian Creoles. Diabetologia 35:632-38.
Clement K, Pueyo M E, Vaxillaire M, Rakotoambinina B, Thuillier F, Passa P, Froguel P, Roberts J, Velho G. 1996. Assessment of insulin sensitivity in glucokinase-deficient subjects. Diabetologia 39:82-90.
Cole S P, Campling B G, Atlaw T, Kozbor D, Roder J C. 1984. Human monoclonal antibodies. Mol Cell Biochem. 62:109-20.
Damcott C M, Moffett S P, Feingold E, Barmada M M, Marshall J A, Hamman R F, Ferrell R E. 2004. Genetic variation in fatty acid-binding protein-4 and peroxisome proliferator-activated receptor gamma interactively influence insulin sensitivity and body composition in males. Metabolism. 53:303-9.
Dandona P, Aljada A, Bandyopadhyay A. 2004. Inflammation: the link between insulin resistance, obesity and diabetes. Trends Immunol. 25:4-7.
Deeb S S, Fajas L, Nemoto M, Pihlajamaki J, Mykkanen L, et al. 1998. A Pro12Ala substitution in PPARgamma2 associated with decreased receptor activity, lower body mass index and improved insulin sensitivity. Nat. Genet. 20:284-87.
Demenais F, Kanninen T, Lindgren C M, Wiltshire S, Gaget S, Dandrieux C, Almgren P, Sjogren M, Hattersley A, Dina C, Tuomi T, McCarthy M I, Froguel P, Groop L C. 2003. A meta-analysis of four European genome screens (GIFT Consortium) shows evidence for a novel region on chromosome 17p11.2-q22 linked to type 2 diabetes. Hum Mol Genet. 12:1865-73.
Dickson L M, Rhodes C J. 2004. Pancreatic beta-cell growth and survival in the onset of type 2 diabetes: a role for protein kinase B in the Akt? Am J Physiol Endocrinol Metab. 287:E192-8.
Eriksson J G, Lindi V, Uusitupa M, Forsen T J, Laakso M, Osmond C, Barker D J. 2002. The effects of the Pro12Ala polymorphism of the peroxisome proliferator-activated receptor-gamma2 gene on insulin sensitivity and insulin metabolism interact with size at birth. Diabetes. 51:2321-4.
Eronen L, Geerts F, Toivonen H. 2004. A Markov chain approach to reconstruction of long haplotypes. Pac Symp Biocomput.:104-15.
Florez J C, Hirschhorn J, Altshuler D. 2003. The inherited basis of diabetes mellitus: implications for the genetic analysis of complex traits. Annu Rev Genomics Hum Genet 4:257-91.
Froguel P, Vaxillaire M, Sun F, Velho G, Zouali H, Butel M O, Lesage S, Vionnet N, Clement K, Fougerousse F, et al. 1992. Close linkage of glucokinase locus on chromosome 7p to early-onset non-insulin-dependent diabetes mellitus. Nature 356:162-164.
Gloyn A L, Hashim Y, Ashcroft S J, Ash-field R, Wiltshire S, Turner R C. 2001. Association studies of variants in promoter and coding regions of beta-cell ATP-sensitive K-channel genes SUR1 and Kir6.2 with Type 2 diabetes mellitus (UKPDS 53). Diabetes Med. 18:206-12.
Gloyn A L, Weedon M N, Owen K R, Turner M J, Knight B A, et al. 2003. Large-scale association studies of variants in genes encoding the pancreatic {beta}-cell KATP channel subunits Kir6.2 (KCNJ11) and SUR1 (ABCC8) confirm that the KCNJ11 E23K variant is associated with type 2 diabetes. Diabetes 52:568-72.
Gough S C, Saker P J, Pritchard L E, Merriman T R, Merriman M E, et al. 1995. Mutation of the glucagon receptor gene and diabetes mellitus in the UK: association or founder effect? Hum. Mol. Genet. 4:1609-12.
Griffiths A D, Malmqvist M, Marks J D, Bye J M, Embleton M J, McCafferty J, Baier M, Holliger K P, Gorick B D, Hughes-Jones N C, et al. 1993. Human anti-self antibodies with high specificity from phage display libraries. EMBO J. 12:725-34.
Gunton J E, Kulkarni R N, Yim S, Okada T, Hawthorne W J, Tseng Y H, Roberson R S, Ricordi C, O'Connell P J, Gonzalez F J, Kahn C R. 2005. Loss of ARNT/HIF1 beta mediates altered gene expression and pancreatic-islet dysfunction in human type 2 diabetes.
Cell. 122: 337-49.
Guthrie R A, Guthrie D W. 2004. Pathophysiology of diabetes mellitus. Crit Care Nurs Q. 27:113-25.
Hager J, Hansen L, Vaisse C, Vionnet N, Philippi A, et al. 1995. A missense mutation in the glucagon receptor gene is associated with non-insulin-dependent diabetes mellitus. Nat. Genet. 9:299-304.
Hani E H, Boutin P, Durand E, Inoue H, Permutt M A, et al. 1998. Missense mutations in the pancreatic islet beta cell inwardly rectifying K+ channel gene (KIR6.2/BIR): a meta-analysis suggests a role in the polygenic basis of Type II diabetes mellitus in Caucasians. Diabetologia 41:1511-15.
Hani E H, Clement K, Velho G, Vionnet N, Hager J, et al. 1997. Genetic studies of the sulfonylurea receptor gene locus in NIDDM and in morbid obesity among French Caucasians. Diabetes 46:688-94.
Hansen T, Echwald S M, Hansen L, Moller A M, Almind K, et al. 1998. Decreased tolbutamide-stimulated insulin secretion in healthy subjects with sequence variants in the high-affinity sulfonylurea receptor gene. Diabetes 47: 598-605.
Hara K, Okada T, Tobe K, Yasuda K, Mori Y, et al. 2000. The Pro12Ala poly-morphism in PPAR gamma2 may confer resistance to type 2 diabetes. Biochem. Biophys. Res. Commun. 271:212-16.
Hay B N, Sorge J A, Shopes B. 1992. Bacteriophage cloning and Escherichia coli expression of a human IgM Fab. Hum Antibodies Hybridomas. 3:81-5.
Hayashi N, Kipriyanov S, Fuchs P, Welschof M, Dorsam H, Little M. 1995. A single expression system for the display, purification and conjugation of single-chain antibodies. Gene. 160:129-30.
Hering S, Karawajew L, Pasternak G. 1988. Raji-K562 hybrids and their use for trioma production. Biomed Biochim Acta. 47:211-6.
Herman W H, Fajans S S, Oritz F J, Smith M J, Sturis J, Bell G I, Polonsky K S, Halter J B. 1994. Abnormal insulin secretion, not insulin resistance, is the genetic or primary defect of MODY in the RW pedigree. Diabetes 43:40-46.
Huse W D, Sastry L, Iverson S A, Kang A S, Alting-Mees M, Burton D R, Benkovic S J, Lerner R A. 1989. Generation of a large combinatorial library of the immunoglobulin repertoire in phage lambda. Science. 246:1275-81.
Inoue H, Ferrer J, Welling C M, Elbein S C, Hoffman M, et al. 1996. Sequence variants in the sulfonylurea receptor (SUR) gene are associated with NIDDM in Caucasians. Diabetes 45:825-31.
Kadowaki T, Kadowaki H, Mori Y, Tobe K, Sakuta R, Suzuki Y, Tanabe Y, Sakura H, Awata T, Goto Y, et al. 1994. A subtype of diabetes mellitus associated with a mutation of mitochondrial DNA. N Engl J Med 330:962-968.
Kahn C R, Flier J S, Bar R S, Archer J A, Gorden P, Martin M M, Roth J. 1976. The syndromes of insulin resistance and acanthosis nigricans. N Engl J Med 294:739-745.
Kohler G, Milstein C. 1975. Continuous cultures of fused cells secreting antibody of predefined specificity. Nature 256:495-497.
Kozbor D, Lagarde A E, Roder J C. 1982. Human hybridomas constructed with antigen-specific Epstein-Barr virus-transformed cell lines. Proc Natl Acad Sci USA. 79:6651-5.
Kwok P-Y. 2001. Methods for genotyping single nucleotide polymorphisms. Ann Rev Genomics Hum Genet. 2:235-258.
Laaksonen D E, Lakka T A, Lakka H-M, Nyyssonen K, Rissanen T, Niskanen L K, Salonen J T. 2002. Serum fatty acid composition predicts development of impaired fasting glycaemia and diabetes in middle-aged men. Diabetic Medicine 19:456-64.
Lakka H-M, Oksanen L, Tuomainen T-P, Kontula K, Salonen J T. 2000. The common pentanucleotide polymorphism of the 3′-untranslated region of the leptin receptor gene is associated with serum insulin levels and the risk of type 2 diabetes in non-diabetic men: a prospective case-control study. J Int Med 248: 77-83.
Lehtovirta M, Kaprio J, Grrop L, trombetta M, Bonadonna R C. 2005. Heritability of model-derived parameters of beta cell secretion during intravenous and oral glucose tolerance tests: a study of twins. Diabetologia 48: 1604-13.
Li S R, Baroni M G, Oelbaum R S, Stock J, Galton D J. 1988. Association of genetic variant of the glucose transporter with non-insulin-dependent diabetes mellitus. Lancet 2:368-70.
Lowe W L Jr. Genetics of diabetes mellitus. Kluwer Academic Publishers, 2001.
Luan J, Browne P O, Harding A H, Halsall D J, O'Rahilly S, Chatterjee V K, Wareham N J. 2001. Evidence for gene-nutrient interaction at the PPARgamma locus. Diabetes. 50:686-9.
Martin B C, Warram J H, Krolewski A S, Bergman R N, Soeldner J S, Kahn C R. 1992. Role of glucose and insulin resistance in development of type 2 diabetes mellitus: results of a 25-year follow-up study. Lancet 340:925-9.
McCarthy M I, Hitman G A, Hitchins M, Riikonen A, Stengard J, et al. 1994. Glu-cokinase gene polymorphisms: a genetic marker for glucose intolerance in a cohort of elderly Finnish men. Diabetes Med. 11:198-204.
Memisoglu A, Hu F B, Hankinson S E, Manson J E, De Vivo I, Willett W C, Hunter D J. 2003. Interaction between a peroxisome proliferator-activated receptor gamma gene polymorphism and dietary fat intake in relation to body mass. Hum Mol Genet. 12:2923-9.
Mori H, Ikegami H, Kawaguchi Y, Seino S, Yokoi N, et al. 2001. The Pro12−Ala substitution in PPAR-gamma is associated with resistance to development of diabetes in the general population: possible involvement in impairment of insulin secretion in individuals with type 2 diabetes. Diabetes 50:891-94.
Motoshima H, Wu X, Sinha M K, Hardy V E, Rosato E L, Barbot D J, Rosato F E, Goldstein B J. 2002. Differential regulation of adiponectin secretion from cultured human omental and subcutaneous adipocytes: effects of insulin and rosiglitazone. J Clin Endocrinol Metab. 87:5662-7.
Nielsen P E, Egholm M, Berg R H, Buchardt O. 1991. Sequence-selective recognition of DNA by strand displacement with a thymine-substituted polyamide. Science. 254:1497-500.
Peltonen L, Jalanko A, Varilo T. Molecular genetics of the Finnish disease heritage. 1999. Hum Mol Genet. 8: 1913-23.
Pontiroli A E, Capra F, Veglia F, Ferrari M, Xiang K S, et al. 1996. Genetic contribution of polymorphism of the GLUT1 and GLUT4 genes to the susceptibility to type 2 (non-insulin-dependent) diabetes mellitus in different populations. Acta Diabetologica 33:193-97.
Poulsen P, Kyvik K O, Vaag A, Beck-Nielsen H. 1999. Heritability of type II (non-insulin-dependent) diabetes mellitus and abnormal glucose tolerance—a population-based twin study. Diabetologia 42:139-45.
Prentki M, Joly E, El-Assaad W, Roduit R. 2002. Malonyl-CoA signaling, lipid partitioning, and glucolipotoxicity: role in beta-cell adaptation and failure in the etiology of diabetes. Diabetes. 51 Suppl 3:S405-13.
Ravussin E, Smith S R. 2002. Increased fat intake, impaired fat oxidation, and failure of fat cell proliferation result in ectopic fat storage, insulin resistance, and type 2 diabetes mellitus. Ann N Y Acad Sci. 967:363-78.
Reardon W, Ross R J M, Sweeney M G, Luxon L M, Pembrey M E, Harding A E, Trembath R C. 1992. Diabetes mellitus associated with a pathogenic point mutation in mitochondrial DNA. Lancet 340:1376-1379.
Sajantila A, Salem A H, Savolainen P, Bauer K, Gierig C, Paabo S. 1996. Paternal and maternal DNA lineages reveal a bottleneck in the founding of the Finnish population. Proc Natl Acad Sci USA. 93: 12035-9.
Salonen J T, Lakka T A, Lakka H-M, Valkonen V-P, Everson S A, Kaplan G A. 1998. Hyperinsulinemia is associated with the Incidence of hypertension and dyslipidemia in middle-aged men. Diabetes 47:270-275.
Salonen J T, Malin R, Tuomainen T P, Nyyssonen K, Lakka T A, Lehtimaki T. 1999. Polymorphism in high density lipoprotein paraoxonase gene and risk of acute myocardial infarction in men: prospective nested case-control study. BMJ. 319:487-9.
Salonen J T, Tuomainen T-P, Kontula K. 2000. Role of C282Y mutation in haemochromatosis gene in development of type 2 diabetes in healthy men: prospective cohort study. Brit Med J 320: 1706-1707.
Salonen J T. 1988. Is there a continuing need for longitudinal epidemiologic research? The Kuopio Ischaemic Heart Disease Risk Factor Study. Ann Clin Res 20: 46-50.
Salonen, J T. 2003. The value of founder populations in genetic analysis. Current Drug Discovery pp. 27, 30-31.
Saltiel A R, Kahn C R. 2001. Insulin signalling and the regulation of glucose and lipid metabolism. Nature. 414:799-806.
Scheen A J. 2003. Pathophysiology of type 2 diabetes. Acta Clin Belg. 58:335-41.
Scheen A J. 2004. Pathophysiology of insulin secretion. Ann Endocrinol (Paris). 65:29-36.
Shulman G I. 2000. Cellular mechanisms of insulin resistance. J Clin Invest. 106:171-6.
Syvaqnen A-C. 2001. Accessing genetic variation: Genotyping single nucleotide polymorphisms. Nature Reviews Genetics. 2:930-942.
Takekawa K, Ikegami H, Fukuda M, Ueda H, Kawaguchi Y, et al. 1994. Early-onset type 2 (non-insulin-dependent) diabetes mellitus is associated with glucokinase locus, but not with adenosine deaminase locus, in the Japanese population. Diabetes Res. Clin. Pract. 23:141-46.
Tao T, Tanizawa Y, Matsutani A, Matsubara A, Kaneko T, Kaku K. 1995. HepG2/erythrocyte glucose transporter (GLUT1) gene in NIDDM: a population association study and molecular scanning in Japanese subjects. Diabetologia 38:942-47.
Taylor S I. 1992. Lilly Lecture: molecular mechanisms of insulin resistance: lessons from patients with mutations in the insulin-receptor gene. Diabetes 41:1473-1490.
Toivonen H T, Onkamo P, Vasko K, Ollikainen V, Sevon P, Mannila H, Herr M, Kere J. 2000. Data mining applied to linkage disequilibrium mapping. Am J Hum Genet. 67:133-45.
Tomas E, Tsao T S, Saha A K, Murrey H E, Zhang Cc C, Itani S I, Lodish H F, Ruderman N B. 2002. Enhanced muscle fat oxidation and glucose transport by ACRP30 globular domain: acetyl-CoA carboxylase inhibition and AMP-activated protein kinase activation. Proc Natl Acad Sci USA. 99:16309-13.
Tuomainen T P, Kontula K, Nyyssonen K, Lakka T A, Helio T, Salonen J T. 1999. Increased risk of acute myocardial infarction in carriers of the hemochromatosis gene Cys282Tyr mutation : a prospective cohort study in men in eastern Finland. Circulation. 100:1274-9.
Uimari P, Kontkanen O, Visscher P M, Pirskanen M, Fuentes R, Salonen J T. 2005. Genome-wide linkage disequilibrium from 100,000 SNPs in the East Finland founder population. Twin Res Hum Genet. 8:185-97.
van den Ouwenland J M W, Lemkes H H P J, Ruitenbeek W, Sandkuijl L A, de Vijlder M F, Struyvenberg P A A, van de Kamp, Maassen J A. 1992. Mutation in mitochondrial tRNA (Leu(URR) gene in a large pedigree with maternally transmitted type II diabetes mellitus and deafness. Nature Genet 1:368-371.
Vaxillaire M, Boccio V, Philippi A, Vigouroux C, Terwilliger J, Passa P, Beckman J S, Velho G, Lathrop G M, Froguel P. 1995. A gene for maturity onset diabetes of the young (MODY) maps to chromosome 12q. Nature Genet 9:418-423.
Vionnet N, Stoffel M, Takeda J, Yasuda K, Bell G I, Zouali H, Lesage S, Velho G, Iris F, Passa P, et al. 1992. Nonsense mutation in the glucokinase gene causes early-onset non-insulin-dependent diabetes mellitus. Nature 356:721-722.
World Health Organization and the International Diabetes Federation. 2004. Diabetes Action Now booklet. http://www.who.int/diabetes/actionnow/booklet/en (Accessed 02.07.04)
World Health Organization. 2004. Diabetes Mellitus fact sheet. http://www.who.int/mediacentre/factsheets/fs138/en (Accessed 02.07.04).
Wu X, Hoffstedt J, Deeb W, Singh R, Sedkova N, Zilbering A, Zhu L, Park P K, Arner P, Goldstein B J. 2001. Depot-specific variation in protein-tyrosine phosphatase activities in human omental and subcutaneous adipose tissue: a potential contribution to differential insulin sensitivity. J Clin Endocrinol Metab. 86:5973-80.
Yamagata K, Furuta H, Oda N, Kaisaki P J, Menzel S, Cox N J, Fajans S S, Signorini S, Stoffel M, Bell G I. 1996. Mutations in the hepatocyte factor-4α gene in maturity-onset diabetes of the young (MODY 1). Nature 384:458-460.
Yamagata K, Oda N, Kaisaki P J, Menzel S, Furuta H, Vaxillaire M, Southarm L, Cox R D, Lathrop G M, Boriraj V V, Chen X, Cox N J, Oda Y, Yano H, Le Beau M M, Yamada S, Nishigori H, Takeda J, Fajans S S, Hattersley A T, Iwasaki N, Hansen T, Pedersen O, Polonsky K S, Bell G I. 1996. Mutations in the hepatocyte nuclear factor-1α gene in maturity-onset diabetes of the young (MODY 3). Nature 384:455-458.
Yamauchi T, Kamon J, Minokoshi Y, Ito Y, Waki H, Uchida S, Yamashita S, Noda M, Kita S, Ueki K, Eto K, Akanuma Y, Froguel P, Foufelle F, Ferre P, Carling D, Kimura S, Nagai R, Kahn B B, Kadowaki T. 2002. Adiponectin stimulates glucose utilization and fatty-acid oxidation by activating AMP-activated protein kinase. Nat Med. 8:1288-95.
Zick Y. 2001. Insulin resistance: a phosphorylation-based uncoupling of insulin signaling. Trends Cell Biol. 11:437-41.

1. A method for risk assessment, diagnosis or prognosis of T2D or a T2D related condition in a mammalian subject comprising:
a) providing a biological sample taken from the subject;
b) assessing one or several biomarkers in said sample, wherein said biomarkers are associated with the T2D associated genes set forth in table 13, or with the proteins or polypeptides encoded by said genes, and;
c) comparing biomarker data obtained in step b) to biomarker data from healthy and diseased people to make risk assessment, diagnosis or prognosis of T2D or a T2D related condition.

2. The method according to claim 1, wherein said biomarkers are polymorphic sites residing in genomic regions containing the T2D associated genes set forth in table 13.

3. The method according to claim 1, wherein said biomarkers are selected from SNP markers set forth in tables 1 to 12.

4. The method according to claim 1, wherein said biomarkers are polymorphic sites associated with one or several of the SNP markers set forth in tables 1 to 12.

5. The method according to claim 1, wherein said biomarkers are polymorphic sites being in complete linkage disequilibrium with one or several of the SNP markers set forth in tables 1 to 12.

6. The method according to claim 1, wherein said biomarkers are expression products of one or several of the said T2D associated genes.

7. The method according to claim 6, wherein said biomarkers are transcription products of one or several of the said T2D associated genes.

8. The method according to claim 6, wherein said biomarkers are translation products of one or several of the said T2D associated genes.

9. The method according to claim 1, wherein said biomarkers are measuring biological activities of the polypeptides encoded by one or several of the said T2D associated genes.

10. The method according to claim 1, wherein said biomarkers are measuring biological functions of the polypeptides encoded by one or several of the said T2D associated genes.

11. The method according to claim 1, wherein said biomarkers are metabolites of the polypeptides encoded by one or several of the said T2D associated genes.

12. The method according to claim 1, wherein said biomarkers are associated to endogenous or exogenous modulators of said T2D associated genes, proteins or polypeptides.

13. The method according to claim 1, wherein said biomarkers are a set of antibodies specific to the polypeptides encoded by one or several of the said T2D associated genes.

14. The method according to claim 1, wherein said biomarkers are any combination of biomarkers of the claims 2 to 13.

15. The method according to claim 1, wherein said method is for identifying subjects having altered risk for developing T2D or a T2D related condition.

16. The method according to claim 1, wherein said method is for diagnosing a subtype of T2D in a subject having T2D or a T2D related condition.

17. The method according to claim 1, wherein said method is for selecting efficient and safe T2D therapy to a subject having T2D or a T2D related condition.

18. The method according to claim 1, wherein said method is for monitoring the effect of a therapy administered to a subject having T2D or a T2D related condition.

19. The method according to claim 1, wherein said method is for predicting the effectiveness of a given therapeutic to treat T2D in a subject having T2D or a T2D related condition.

20. The method according to claim 1, wherein said method is for selecting efficient and safe T2D preventative therapy to a subject having increased risk of T2D or a T2D related condition.

21. The method according to claim 1, wherein said method is for monitoring the effect of preventive therapy administered to a subject having increased risk of T2D or a T2D related condition.

22. The method according to claim 1, wherein said method is for predicting the effectiveness of a given therapeutic to prevent T2D in a subject having increased risk of T2D or a T2D related condition.

23. The method according to claim 1 further comprising a marker set to assess the ancestry of a subject.

24. The method according to claim 23, wherein a SNP marker set is used to assess the ancestry of a subject.

25. The method according to claim 23, wherein a microsatellite marker set is used to assess the ancestry of an individual.

26. The method according to claim 1 further comprising a step of combining non-genetic information with the biomarker data to make risk assessment, diagnosis or prognosis of T2D or a T2D related condition.

27. The method according to claim 26, wherein the non-genetic information concerns age, gender, ethnicity, socioeconomic status, history of gestational diabetes, other medical history of the subject, family history of relevant conditions, psychological traits and states, behaviour patterns and habits, biochemical measurements, clinical measurements, and measurements of obesity and adiposity.

28. The method according to claim 27, wherein the other medical history of the subject concerns the metabolic syndrome, glucose intolerance, increased insulin resistance, obesity, nephropathies, hypothyroidism, hyperthyroidism, disorders of the pituitary gland, disorders of the hypothalamus, disorders of the pancreas, appetite and eating disorders and conditions which limit physical activity, low weight at birth and/or premature birth.

29. The method according to claim 27, wherein the relevant family history information concerns type I and type 2 diabetes, gestational diabetes, other type of diabetes, the metabolic syndrome, glucose intolerance, increased insulin resistance, obesity, hypothyroidism, hyperthyroidism, disorders of the pituitary grand, disorders of the hypothalamus, disorders of the pancreas and appetite and eating disorders.

30. The method according to claim 27, wherein the biochemical measurements include measurements of lipids, lipoproteins, carbohydrates and peptides and proteins in human tissues and body fluids.

31. The method according to claim 30, wherein the protein measurements include the measurements of prohormones, hormones, enzymes and receptors.

32. The method according to claims 30 and 31, wherein the measurements include the measurements of glycated peptides and proteins, advanced glycated end products, oxidatively modified proteins and peptides, glucagons, glucagons-like peptides (GLP), other insulinotropic peptides, proinsulins, insulin, insulin degrading enzymes, growth hormone, thyrotropin-releasing hormone (TRH), TRH-like peptides, prolactine, amylins, homocysteine, C-peptide, leptins, adiponectins, ghrelins, gastrins, resistin, obestatin, incretins, markers of mild chronic inflammation, such as TNFα, IL-6 and C-reactive protein, dipeptidyl peptidase IV, endothelins, pituitary adenylate cyclase activating peptides (PACAPs), vasoactive intestinal peptides (VIPs), hypothalamic regulatory peptides, opioid peptides, neuropeptide Y, adrenomedullin, atrial and brain natriuretic peptides (ANPs, BNPs), heat shock protein derived peptides, ferritin, transferrin, ceruloplasmin, albumin, the endogenous activators, inhibitors, inactivators, receptors and degradators of the said peptides and enzymes involved in the synthesis and release of the said peptides.

33. The method according to claim 27, wherein the measurements of obesity and adiposity include height, weight, body-mass index (kg/m2), waist circumference, waist-to-hip circumference ratio, skinfold thickness measurements, adipose tissue thickness measurements and measurements of amount and proportion of adipose tissue of the body.

34. The method according to claim 27, wherein the behaviour patterns and habits include tobacco smoking, physical activity, dietary intakes of nutrients, salt intake, alcohol intake and consumption patterns and coffee consumption and quality.

35. The method according to claim 1 further comprising a step of calculating the risk of T2D using a logistic regression equation as follows:
Risk of T2D=[1+e^{-(a+Σ(bi*Xi)}]⁻¹, where e is Napier's constant, Xᵢ; are variables associated with the risk of T2D, bᵢ; are coefficients of these variables in the logistic function, and a is the constant term in the logistic function.

36. The method according to claim 35, wherein a and bi; are determined in the population in which the method is to be used.

37. The method according to claim 35, wherein Xi are selected among the variables that have been measured in the population in which the method is to be used.

38. The method according to claim 35, wherein Xi are selected among the SNP markers of tables 1 to 12, among haplotypes of tables 2, 3, 4, 5, 6, 7, 9 and 10 and among nongenetic variables of the invention.

39. The method according to claim 35, wherein bᵢ; are between the values of −20 and 20 and/or wherein Xᵢ; can have values between −99999 and 99999 or are coded as 0 (zero) or 1 (one).

40. The method according to claim 35, wherein i are between the values 0 (none) and 100,000.

41. The method according to claim 35, wherein subject's short term, median term, and/or long term risk of T2D is predicted.

42. A test kit based on a method of claim 1 for risk assessment, diagnosis or prognosis of T2D or a T2D related condition comprising:
a) reagents, materials and protocols for assessing type and/or level of one or more biomarkers in a biological sample, wherein said biomarkers are associated to the T2D associated genes set forth in table 13, or to the proteins or polypeptides encoded by said genes, and;
b) instructions and software for comparing the biomarker data from a subject to biomarker data from healthy and diseased people to make risk assessment, diagnosis or prognosis of T2D or a T2D related condition.

43. The kit of claim 42 comprising a PCR primer set for amplifying nucleic acid fragments containing one or several polymorphic sites residing in genomic regions containing the T2D associated genes set forth in table 13.

44. The kit of claim 42 comprising a capturing nucleic acid probe set specifically binding to one or several polymorphic sites residing in genomic regions containing the T2D associated genes set forth in table 13.

45. The test kit of claim 42 comprising a microarray or multiwell plate to assess the genotypes.

46. The test kit according to claim 42 further comprising a questionnaire and instructions for collecting personal and clinical information from the subject, and software and instructions for combining personal and clinical information with biomarker data to make risk assessment, diagnosis or prognosis of T2D or a T2D related condition.

47. The test kit according to claim 46, wherein the non-genetic information concerns age, gender, ethnicity, socioeconomic status, history of gestational diabetes, other medical history of the subject, family history of relevant conditions, psychological traits and states, behaviour patterns and habits, biochemical measurements, clinical measurements, and measurements of obesity and adiposity.

48. The test kit according to claim 47, wherein the other medical history of the subject concerns the metabolic syndrome, glucose intolerance, increased insulin resistance, obesity, nephropathies, hypothyroidism, hyperthyroidism, disorders of the pituitary gland, disorders of the hypothalamus, disorders of the pancreas, appetite and eating disorders and conditions which limit physical activity, low weight at birth and/or premature birth.

49. The test kit according to claim 47, wherein the relevant family history information concerns type 1 and type 2 diabetes, gestational diabetes, other type of diabetes, the metabolic syndrome, glucose intolerance, increased insulin resistance, obesity, hypothyroidism, hyperthyroidism, disorders of the pituitary grand, disorders of the hypothalamus, disorders of the pancreas and appetite and eating disorders.

50. The test kit according to claim 47, wherein the biochemical measurements include measurements of lipids, lipoproteins, carbohydrates and peptides and proteins in human tissues and body fluids.

51. The test kit according to claim 50, wherein the protein measurements include the measurements of prohormones, hormones, enzymes and receptors.

52. The test kit according to claims 50 and 51, wherein the measurements include the measurements of glycated peptides and proteins, advanced glycated end products, oxidatively modified proteins and peptides, glucagons, glucagons-like peptides (GLP), other insulinotropic peptides, proinsulins, insulin, insulin degrading enzymes, growth hormone, thyrotropin-releasing hormone (TRH), TRH-like peptides, prolactine, amylins, homocysteine, C-peptide, leptins, adiponectins, ghrelins, gastrins, resistin, obestatin, incretins, markers of mild chronic inflammation, such as TNFα, IL-6 and C-reactive protein, dipeptidyl peptidase IV, endothelins, pituitary adenylate cyclase activating peptides (PACAPs), vasoactive intestinal peptides (VIPs), hypothalamic regulatory peptides, opioid peptides, neuropeptide Y, adrenomedullin, atrial and brain natriuretic peptides (ANPs, BNPs), heat shock protein derived peptides, ferritin, transferrin, ceruloplasmin, albumin, the endogenous activators, inhibitors, inactivators, receptors and degradators of the said peptides and enzymes involved in the synthesis and release of the said peptides.

53. The test kit according to claim 47, wherein the measurements of obesity and adiposity include height, weight, body-mass index (kg/m2), waist circumference, waistto-hip circumference ratio, skinfold thickness measurements, adipose tissue thickness measurements and measurements of amount and proportion of adipose tissue of the body.

54. The test kit according to claim 47, wherein the behaviour patterns and habits include tobacco smoking, physical activity, dietary intakes of nutrients, salt intake, alcohol intake and consumption patterns and coffee consumption and quality.

55. The test kit according to claim 42, wherein said biomarkers are polymorphic sites residing in genomic regions containing the T2D associated genes set forth in table 13.

56. The test kit according to claim 42, wherein said biomarkers are selected from SNP markers set forth in tables 1 to 12.

57. The test kit according to claim 42, wherein said biomarkers are polymorphic sites associated with one or several of the SNP markers set forth in tables 1 to 12.

58. The test kit according to claim 42, wherein said biomarkers are polymorphic sites being in complete linkage disequilibrium with one or several of the SNP markers set forth in tables 1 to 12.

59. The test kit according to claim 42, wherein said biomarkers are expression products of one or several of the said T2D associated genes.

60. The test kit according to claim 59, wherein said biomarkers are transcription products of one or several of the said T2D associated genes.

61. The test kit according to claim 59, wherein said biomarkers are translation products of one or several of the said T2D associated genes.

62. The test kit according to claim 42, wherein said biomarkers are measuring biological activities of the polypeptides encoded by one or several of the said T2D associated genes.

63. The test kit according to claim 42, wherein said biomarkers are measuring biological functions of the polypeptides encoded by one or several of the said T2D associated genes.

64. The test kit according to claim 42, wherein said biomarkers are metabolites of the polypeptides encoded by one or several of the said T2D associated genes.

65. The test kit according to claim 42, wherein said biomarkers are associated to endogenous or exogenous modulators of said T2D associated genes, proteins or polypeptides.

66. The test kit according to claim 42, wherein said biomarkers are a set of antibodies specific to the polypeptides encoded by one or several of the said T2D associated genes.

67. The test kit according to claim 42, wherein said biomarkers are any combination of biomarkers tised in the test ]iits of the claims 55 to 66.

68. The test kit according to claim 42, wherein said kit is for identifying subjects having altered risk for developing T2D or a T2D related condition.

69. The test kit according to claim 42, wherein said kit is for diagnosing a subtype of T2D in a subject having T2D or a T2D related condition.

70. The test kit according to claim 42, wherein said kit is for selecting efficient and safe T2D therapy to a subject having T2D or a T2D related condition.

71. The test kit according to claim 42, wherein said kit is for monitoring the effect of a therapy administered to a subject having T2D or a T2D related condition.

72. The test kit according to claim 42, wherein said kit is for predicting the effectiveness of a given therapeutic to treat T2D in a subject having T2D or a T2D related condition.

73. The test kit according to claim 42, wherein said kit is for selecting efficient and safe T2D preventative therapy to a subject having increased risk of T2D or a T2D related condition.

74. The test kit according to claim 42, wherein said kit is for monitoring the effect of preventive therapy administered to a subject having increased risk of T2D or a T2D related condition.

75. The test kit according to claim 42, wherein said kit is for predicting the effectiveness of a given therapeutic to prevent T2D in a subject having increased risk of T2D or a T2D related condition.

76. The test kit of claim 42 further comprising a marker set to assess the ancestry of a subject.

77. The test kit according to claim 76, wherein a SNP marker set is used to assess the ancestry of a subject.

78. The test kit according to claim 76, wherein a microsatellite marker set is used to assess the ancestry of an individual.

79. A method for preventing or treating T2D or a T2D related condition in a mammalian subject comprising a therapy modulating biological activity or function of a protein or a polypeptide encoded by a T2D associated gene set forth in table 13.

80. The method according to claim 79 comprising administering to a mammalian subject in need of such treatment an effective amount of a therapeutic agent enhancing or reducing expression of one or several T2D associated genes set forth in table 13.

81. The method according to claim 79 comprising administering to a mammalian subject in need of such treatment an effective amount of a therapeutic agent enhancing or reducing biological activity or function of a protein or a polypeptide encoded by a T2D associated gene set forth in table 13.

82. The method according to claim 79 comprising administering to a mammalian subject in need of such treatment an effective amount of a therapeutic agent enhancing or reducing expression of one or several genes in biological networks and/or metabolic pathways related to a protein or to a polypeptide encoded by a T2D associated gene set forth in table 13.

83. The method according to claim 79 comprising administering to a mammalian subject in need of such treatment an effective amount of a therapeutic agent enhancing or reducing activity of one or several biological networks and/or metabolic pathways related to a protein or to a polypeptide encoded by a T2D associated gene set forth in table 13.

84. The method according to claim 79 comprising administering to a mammalian subject in need of such treatment an effective amount of a therapeutic agent enhancing or reducing activity of one or several pathophysiological pathways involved in T2D or a related condition and related to polypeptides encoded by a T2D associated gene set forth in table 13.

85. The method according to claim 79 comprising a therapy restoring, at least partially, the observed alterations in biological activity of one or several proteins or polypeptides encoded by a T2D associated gene set forth in table 13 in said subject, when compared with T2D free healthy subjects.

86. The method according to claim 79 comprising a therapy restoring, at least partially, the observed alterations in expression of one or several T2D associated genes set forth in table 13 in said subject, when compared with T2D free healthy subjects.

87. The method according to claim 79 comprising gene therapy or gene transfer.

88. The method according to claim 87, wherein said therapy comprises the transfer of one or several T2D associated genes set forth in table 13 or variants, fragments or derivatives thereof.

89. The method according to claim 88, wherein said T2D associates genes set forth in table 13 or variants, fragments or derivatives thereof are associated with reduced risk of T2D or a T2D related condition.

90. The method according to claim 87, wherein said therapy comprises treating regulatory regions and/or gene containing region of one or several T2D risk genes set forth in table 13 or variants, fragments or derivatives thereof in somatic cells, in stem cells, or in affected tissues of said subject.

91. The method according to claim 79, wherein said therapy comprises a recombinant polypeptide encoded by a T2D risk gene set forth in table 13 or variant, fragment or derivative thereof.

92. The method according to claim 79, wherein said therapy comprises an antibody binding to a proteins or to a polypeptide encoded by a T2D risk gene set forth in table 13.

93. The method according to claim 79, wherein said therapy comprises an agent binding to a protein or to a polypeptide encoded by a T2D associated gene set forth in table 13.

94. The method according to claim 79, wherein said therapy is based on sequence specific gene silencing agents such as siRNA hybridising to mRNA and/or to hnRNA of a T2D associated gene set forth in table 13.

95. The method according to claim 79, wherein said therapy is based on sequence specific gene silencing agents such as siRNA hybridising to mRNA and/or to hnRNA of one or several genes in biological networks and/or metabolic pathways related to proteins and polypeptides encoded by said T2D associated genes set forth in table 13.

96. The method according to claim 79, wherein said therapy is a dietary treatment or a vaccination.

97. A method for screening agents useful in prevention or treatment of T2D or a T2D related condition comprising determining the effect of agents either on biological networks related to one or several polypeptides encoded by one or-several T2D associated genes set forth in table 13 or on metabolic pathways related to one or several polypeptides encoded by said T2D associated genes in living cells; wherein an agent altering activity of one or several said biological networks and/or metabolic pathways is considered useful in prevention or treatment of T2D or a T2D related condition.

98. The method according to claim 97, wherein the candidate agents are administered to a model system or organism, wherein agents altering or modulating transcriptional expression, translation, biological activity, biological structure, and/or amount of metabolites of one or several of the said T2D associated genes or polypeptides are considered useful in prevention or treatment of T2D or a T2D related condition.

99. The method according to claim 98, wherein the model system or organism is a non-human transgenic animal expressing one or several of the T2D associated genes set forth in table 13.

100. The method according to claim 98 comprising cultured microbial, insect or mammalian cells expressing one or several of the T2D associated genes set forth in table 13.

101. The methods according to claim 98 comprising mammalian tissues, organs or organ systems expressing one or several of the T2D associated genes set forth in table 13.

102. A method of using non-human transgenic animals expressing one or several of the T2D associated genes set forth in table 13 to study pathophysiology and/or molecular mechanisms involved in T2D or a T2D related condition.

103. A pharmaceutical composition useful in prevention or treatment of T2D or a T2D related condition comprising an agent altering biological activity or function of one or several polypeptides encoded by one or several T2D associated genes set forth in table 13.

104. The pharmaceutical composition according to claim 103 comprising an agent altering expression of one or several T2D associated genes set forth in table 13.

105. The pharmaceutical composition according to claim 103 comprising an agent altering expression of one or several genes in biological networks and/or metabolic pathways related to polypeptides encoded by a T2D associated gene set forth in table 13.

106. The pharmaceutical composition according to claim 103 comprising an agent altering activity of one or several biological networks and/or metabolic pathways related to polypeptides encoded by a T2D associated gene set forth in table 13.

107. The pharmaceutical composition according to claim 103 comprising an agent altering activity of one or several pathophysiological pathways involved in T2D or in a T2D related condition, and related to polypeptides encoded by a T2D associated gene set forth in table 13.

108. The pharmaceutical composition according to claim 103 comprising an agent restoring, at least partially, the observed alterations in biological activity of one or several polypeptides encoded by a T2D associated gene set forth in table 13 in said subject, when compared with T2D free healthy subjects.

109. The pharmaceutical composition according to claim 103 comprising an agent restoring, at least partially, the observed alterations in expression of one or several T2D associated genes set forth in table 13 in said subject, when compared with T2D free healthy subjects.

110. The pharmaceutical composition according to claim 103 comprising a polynucleotide hybridising either to a mRNA or to a regulatory region of a T2D associated gene set forth in table 13.

111. The pharmaceutical composition according to claim 103 comprising a recombinant polypeptide encoded by a T2D risk gene set forth in table 13 or variant, fragment or derivative thereof.

112. The pharmaceutical composition according to claim 103 comprising an antibody binding to one or several polypeptides encoded by a T2D risk gene set forth in table 13.

113. The pharmaceutical composition according to claim 103 comprising an agent binding to one or several polypeptides encoded by a T2D associated gene set forth in table 13.

114. The pharmaceutical position according to claim 103 comprising sequence specific gene silencing agents such as siRNA hybridising to mRNA and/or to hnRNA of a T2D associated gene set forth in table 13.

115. The pharmaceutical composition according to claim 103 comprising sequence specific gene silencing agents such as siRNA hybridising to mRNA and/or to hnRNA of one or several genes in biological networks and/or metabolic pathways related a protein or to a polypeptide encoded by a T2D associated gene set forth in table 13.
